(19) United States
(12) Patent Application Publication
(10) Pub. No.: US 2017/0231561 A1
(43) Pub. Date: Aug. 17, 2017

(54) DRUG EFFICACY EVALUATION ASSISTING SYSTEM, AND DRUG EFFICACY EVALUATION ASSIST INFORMATION PRESENTING METHOD

(71) Applicants: Hitachi, Ltd., Tokyo (JP); JICHI MEDICAL UNIVERSITY, Shimotsuke-shi (JP)

(72) Inventors: Stephanie SUTOKO, Tokyo (JP); Tsukasa FUNANE, Tokyo (JP); Hiroki SATO, Tokyo (JP); Atsushi MAKI, Tokyo (JP); Yukifumi MONDEN, Shimotsuke-shi (JP); Masako NAGASHIMA, Shimotsuke-shi (JP); Ippeita DAN, Shimotsuke-shi (JP); Takanori YAMAGATA, Shimotsuke-shi (JP)

(21) Appl. No.: 15/409,944

(22) Filed: Jan. 19, 2017

(30) Foreign Application Priority Data
Feb. 12, 2016 (JP) 2016-024913

(51) Int. Cl. A61B 5/00; A61B 5/0476; A61B 5/04

(52) U.S. Cl. CPC A61B 5/4848; A61B 5/04012; A61B 5/0476; A61B 5/742; A61B 2560/0475

(57) ABSTRACT

Provided herein is a technique for effectively and quantitatively evaluating the symptom improving effect of a treatment given to a subject (patient). The invention provides a technique for diagnosing, evaluating, monitoring, and predicting drug efficacy in individuals (patients) with possible mental disorders such as ADHD, autism, and depression. Specifically, patient's data are simultaneously analyzed using several variables, such as biological measurements (e.g., brain activity measurements) and cognitive performance assessments, involving, for example, a patient (dependent variable), a medication type and dose (independent variables), a diagnosis profile score (DSM) and a rating scale (manifest variables), and an efficacy index (a predictor variable of a future treatment).

### TECHNICAL FIELD

The present invention relates to a drug efficacy evaluation assisting system, and a drug efficacy evaluation assist information presenting method.

### BACKGROUND ART

Attention deficit and hyperactivity disorder (ADHD) is a typical brain function disorder marked by core symptoms including inattention, hyperactivity, and impulsivity. Traditionally, a typical diagnosis of ADHD involves monitoring behaviors, and the diagnosis is often made subjectively by the doctor. Behavior observation is also usually relied upon for the evaluation of ADHD medications such as methylphenidate (MPH) sustained-release agents, and atomoxetine (ATX) with regard to how these drugs work in the brain or if these drugs are actually working. Decisions such as choosing or changing drugs, and determining the oral dosage are also based on behavior observation in many cases.

However, evaluations based on behavior observation are subjective to the views of an observer. This has created a demand for development of a method that can be used to make a more objective diagnosis or to objectively evaluate therapeutic effects through visualization of the characteristic brain function changes of ADHD. For example, PTL 1 discloses finding an index by comparing brain disorder patients and healthy individuals. NPL 1 has shown drug-specific brain-function (brain activity amplitude) recovery effects through studies of different drugs, and measured brain activity patterns by near-infrared spectroscopy (NIRS).

### CITATION LIST

### Patent Literature

PTL 1: US Patent Application 2005/0273017

### Non Patent Literature

NPL 1: M. Nagashima, Y. Monden, I. Dan, H. Dan, T. Mizutani, D. Tsuzuki, Y. Kyutoku, Y. Gunji, D. Hirano, T. Taniguchi, H. Shimoizumi, M. Y. Momoi, T. Yamagata, E. Watanabe., Neuropharmacological effect of atomoxetine on attention network in children with attention deficit hyperactivity disorder during oddball paradigms as assessed using functional near-infrared spectroscopy, Neurophotonics 1(2), 025007 (2014)

### SUMMARY OF INVENTION

### Technical Problem

However, the method of PTL 1 does not compare brain disorder patients. Specifically, no consideration is given to the effects of drugs on brain disorder patients. It is accordingly not possible to find an index of drug-specific brain-function recovery effect as taught in NPL 1.

The invention was made under these circumstances, and the invention is intended to provide a technique that enables more effective and quantitative evaluations of the symptom improving effect of a treatment on a subject (patient).

### Solution to Problem

In order to find a solution to the foregoing problems, the invention proposes a drug efficacy evaluation assisting system for assisting evaluation of the efficacy of a drug treatment on a subject of interest. In the system, brain activity information measured for a subject of interest before and after drug administration is read from a memory storing the brain activity information of a plurality of subjects, including the subject of interest, measured before and after drug administration. Here, the memory stores the brain activity information with corresponding measurement numbers. The system then calculates modulation of the brain activity before and after drug administration for each measurement number. The relationship between the measurement number and the brain activity modulation of the subject of interest is then displayed on a screen of a display device.

Other features of the invention will be more clearly understood from the descriptions of the specification and the accompanying drawings. Embodiments of the invention are achieved and accomplished with elements and combinations of different elements, along with the detailed descriptions below, and the form of the claims set forth below.

It is to be understood that the descriptions of the specification serve solely to illustrate the typical examples of the invention, and are not intended to limit the claims, or the implementations of the invention in any ways.

### Advantageous Effects of Invention

The invention enables effective, objective, and quantitative evaluations of the symptom improve effect of a treatment such as drug administration. The invention also enables assisting doctors and operators to comprehensively determine the presence or absence of efficacy. The invention also can assist patients and patient's families to appropriately choose drugs.

### BRIEF DESCRIPTION OF DRAWINGS

FIG. 1 is a diagram representing a schematic structure of a drug efficacy evaluation assisting system (also referred to as diagnosis assisting device, diagnosis assisting system, or treatment evaluation system) 1 according to an embodiment of the invention.

FIG. 2 is a diagram representing exemplary structures of databases in memory 109.

FIG. 3 is a diagram representing an example of the way that a measurement probe 300 is fitted to the head of a subject (patient) for the measurement of biological signals (brain signals) from patient.

FIG. 4 is a diagram representing a data sequence in the drug efficacy evaluation assisting system 1 of the embodiment.

FIG. 5 is a diagram showing an exemplary structure of a channel selecting display screen 500 of the embodiment.

FIG. 6 is a diagram showing details of hemoglobin waveform 600 at the selected channel before and after drug administration.

FIG. 7 is a diagram showing an exemplary structure of a GUI 700 that a doctor or other user uses to enter an analysis-prediction command.

FIG. 8 is a flowchart explaining an overview of the processes by the drug efficacy program 105 of the embodiment.

FIG. 9 is a diagram showing examples of a list display of the efficacy analysis result of the embodiment.

FIG. 10A is a flowchart explaining details of the efficacy index computation (step 803) of the embodiment (first half of the process).

FIG. 10B is a flowchart explaining details of the efficacy index computation (step 803) of the embodiment (second half of the process).

FIG. 11 is a flowchart explaining details of the efficacy index computation by activity-variability analysis (step 1003).

FIG. 12 is a graph 1200 representing the efficacy index of Hb change (simple modulation) (a diagram representing efficacy for the patient (subject) of interest against a comparison group).

FIG. 13 shows a scatter chart 1300 sorting efficacy by Hb change (z-score).

FIG. 14 shows a scatter chart 1400 sorting efficacy by Hb change (z-score contrast).

FIG. 15 shows a scatter chart 1500 sorting efficacy by activity-variability analysis (clustering).

FIG. 16 shows a diagram 1600 representing the relationship between task correctness change and blood volume change.

FIG. 17 shows a correlation diagram used to grasp the intensity of the functional connectivity between channels.

FIG. 18A is a flowchart explaining details of the response prediction process (step 811) for predicting a response to a future treatment (first half of the process).

FIG. 18B is a flowchart explaining details of the response prediction process (step 811) for predicting a response to a future treatment (second half of the process).

FIG. 19 is a diagram representing a dose-index relation 1900 generated in the response prediction process (S1802 to S1808 in FIG. 18).

FIG. 20 shows the probability generated in the response prediction process (steps 1811 and 1812) plotted in the dose-index relation 2000 (probability analysis result).

FIG. 21 is a diagram representing an exemplary structure of a GUI showing a report displayed when the response prediction process cannot be executed as instructed by the prediction command (S1815 in FIG. 18).

FIG. 22 is a diagram representing an example of the relationship 2200 between efficacy index and measurement number generated in the response prediction process (S1818 to S1820 in FIG. 18: sigmoid prediction).

FIG. 23 is a diagram representing an example of the relationship 2300 between variables generated in the response prediction process (S1821 to S1825 in FIG. 18: prediction by multiple linear regression).

FIG. 24 is a diagram representing an example of a displayed ANOVA (analysis of variance) result.

### DESCRIPTION OF EMBODIMENTS

An embodiment of the invention provides a technique for diagnosing, evaluating, monitoring, and predicting efficacy on individuals (patients) with possible mental disorders such as ADHD, autism, depression. In the present embodiment, patient's data are simultaneously analyzed using several variables, such as biological measurements (e.g. brain activity measurements) and cognitive performance assessments, involving, for example, a patient (dependent variable), a medication type and dose (independent variables), a diagnosis profile score (DSM) and a rating scale (manifest variables), and an efficacy index (predictor variable of future treatment).

The embodiment of the invention is described below with reference to the accompanying drawings. In the drawings, the same reference numerals may be used to refer to functionally the same elements. The drawings represent specific embodiments and specific examples of implementation based on the principle of the invention. However, these embodiments and examples are intended to help understand the invention, and should not be used to narrowly interpret the invention.

The embodiment below is described in sufficient detail so that a skilled person can implement the invention. However, it is to be understood that other implementations and forms are possible, and that various alterations may be made to the configurations and structures, and replacements of various elements are possible within the technical scope and the spirit of the invention. Accordingly, the descriptions below should not be construed to be limiting.

The embodiment of the invention may be implemented as software that operates on an all-purpose computer, or as designated hardware. Implementations based on a combination of software and hardware are also possible.

In the descriptions of the invention below, information will be described in tabular form. However, the information is not necessarily required to be in the form of a tabular data structure, and may be expressed in other forms, including data structures such as a list, DB (database), and queues. As such, data structures such as tables, lists, DB, and queues are also referred to simply as “information” to indicate that the information is not dependent on data structure.

In describing the content of information, the content may be expressed by using terms such as “identification information”, “identifier”, “designation”, “name”, and “ID”, and these are interchangeable.

In the descriptions below, “prescription” and “administered drug” have the same meaning, and are interchangeable. Similarly, the terms “prescription dose”, “applied dose”, “dosage”, and “dose” have the same meaning, and are interchangeable.

### Configuration of Drug Efficacy Evaluation Assisting System

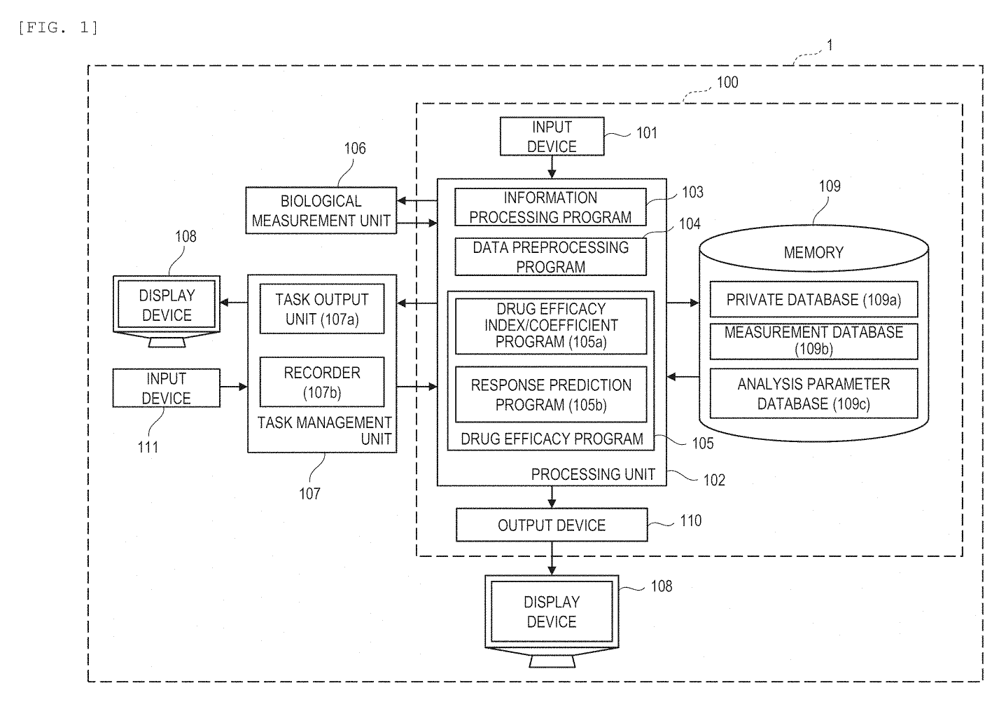

FIG. 1 is a diagram representing a schematic structure of a drug efficacy evaluation assisting system (also referred to as diagnosis assisting device, diagnosis assisting system, or treatment evaluation system) 1 according to the embodiment of the invention.

The drug efficacy evaluation assisting system 1 includes an evaluation device 100, a biological measurement unit 106, a task (challenge or stimulation) management unit 107 for controlling the presentation of tasks in a cognitive test, a display device 108 for displaying the evaluation result of the evaluation device 100, an input device 111 that a patient uses to enter, for example, an answer or a response to a cognitive test (e.g., an attention task, an inhibition task, and a working memory test), and a display device 102 that displays, for example, a problem for a cognitive test, and presents it to a patient. The task management unit 107 includes a task output unit 107a that executes a process for outputting a task, and a recorder 107b for recording a patient's response to a task. The patient's response to a task is sent to a processing unit 102. Here, tasks are given as an attention task, an inhibition task, and a working memory test, as an example. It is, however, possible to use other tasks.

The evaluation device 100 includes a memory 109 storing various databases, an input device 101 that a doctor or an operator uses to enter various types of information (e.g., patient information, and a manifest variable), various data (e.g., a non-parameter variable), and commands (e.g., an analysis command, and a data acquisition command), the processing unit (processor) 102 that is connected to the input device 101, the biological measurement unit 106, the task (stimulation) management unit 107, and the memory 109, and that processes data (e.g., measurement results from the biological measurement unit 106, results of cognitive tests, and data from the databases) or information from these components according to various programs, and an output device 110 that executes processes for displaying, for example, analysis results, and prediction results on the display device 108.

The memory 109 has a private database 109a that stores data and information of patients, a measurement database (population data) 109b that stores biometric data (measured brain signals of patients), and an analysis parameter database 109c that stores analysis parameters.

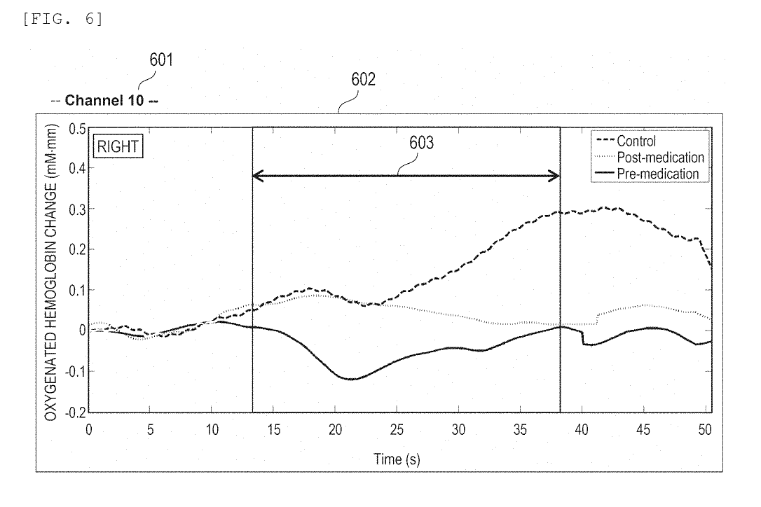

The processing unit 102 executes various programs, specifically, an information processing program 103, a data preprocessing program 104, and a drug efficacy program 105 that includes a drug efficacy index/coefficient program 105a, and a response prediction program 105b. The processing unit 102 processes and analyzes biological signals and task result data, using the data preprocessing program 104, and the drug efficacy program 105. For analysis of biometric signals and task result data, the processing unit 102 may compare these signals and data with the data stored in the private database 109a and the measurement database 109b of the memory 109, or may use the analysis parameters of analyzed signals stored in the analysis parameter database 109c, and a drug efficacy evaluation index. Here, drug efficacy is quantitatively defined as an efficacy index (see, for example, FIG. 7 for the index).

The drug efficacy index/coefficient program 105a uses the current measurement data, and the stored data in the memory, and converts the measurement data into an index. Whether to use which index is entered by a doctor or an operator via the input device 101, as will be described later with reference to FIG. 7.

In order to obtain information about drug administration and treatment for individual patients, the response prediction program 105b estimates efficacy (efficacy changes) at a personal level according to the index selected by a doctor or an operator. The output device 110 processes the analysis result in a manner that varies with a form of display, and displays it on the screen of the display device 108.

### Database Structure

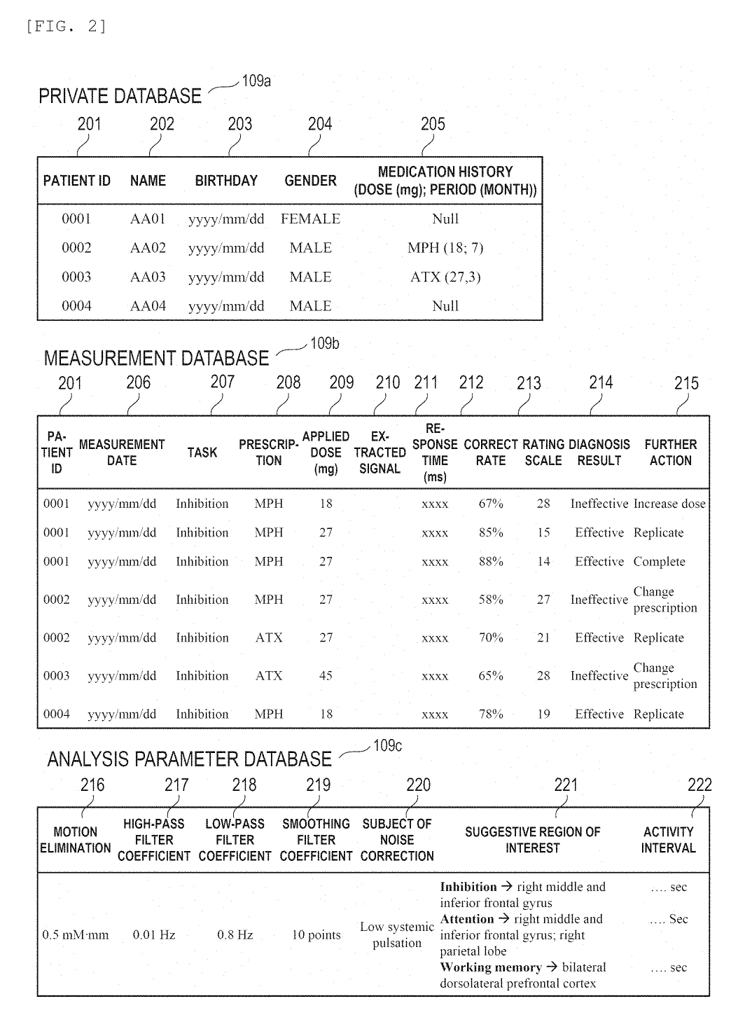

FIG. 2 is a diagram representing exemplary structures of databases in the memory 109.

The private database 109a stores personal information of patients, and is configured to include items such as a patient ID 201, name 202, birthday 203, gender 204, and medication history 205 with which patients can be uniquely specified or identified. Here, the medication history 205 represents information including the type and dose of administered drug, and the duration of administration. Null indicates that there is no history, i.e., the patient has undertaken the treatment for the first time. Here, the descriptions are given for patients. However, the memory 109 may store information of healthy individuals. In this way, the personal data of patients can be compared with data of healthy individuals.

The measurement database 109b stores data associated with measurement data, and is configured to include items such as a patient ID 201, a measurement date 206, a task 207 indicative of the type of the task the patient has undertaken, a prescription 208 indicative of the type of administered drug, an applied dose 209, an extracted signal 210 obtained by extracting a part of biometric signal, a response time 211 indicative of the patient's response time, a correct rate 212 representing task correctness, a rating scale 213 of rating evaluation (for example, a subjective rating evaluation resulting from observation of patient's conditions by a caregiver; information representing a non-parameter variable), a diagnosis result 214 indicative of the presence or absence of efficacy, and a further action 215. The diagnosis result 214 is information indicating that, for example, efficacy is present (Effective), efficacy is absent (Ineffective)”, and efficacy is unclear (Unclear). The further action 215 represents information that indicates to, for example, increase dose (Increase dose), continue using the same dose (Replicate), end administration (Complete), and change prescription (Change prescription). When the diagnosis result 214 is “Ineffective”, a doctor or an operator can predict how the patient will respond to a different prescription or a different dose (treatment result is predicted), using the response prediction program 105b. When the diagnosis result is “Effective”, a doctor or an operator can reevaluate the stability and/or reliability of efficacy by repeating the same measurement on another day, or can end the evaluation upon deciding a drug that is suited for the patient.

The analysis parameter database 109c stores the latest update of optimization preprocessing parameter to be used by the data preprocessing program 104, and is configured to include items such as motion elimination 216 indicative of the amplitude value for removing motion, high-pass filter coefficient 217 for removing a low-frequency component of a biometric signal, low-pass filter coefficient 218 for removing a high-frequency component of a biometric signal, smoothing coefficient 219 indicating the coefficient of a smoothing filter, subject of noise correction 220 indicative of the subject for which noise is corrected, suggestive region of interest 221 indicative of a signal acquisition region, and activity interval 222 indicative of a signal extraction interval, which is determined by the type of task.

### Example of Probe Placement

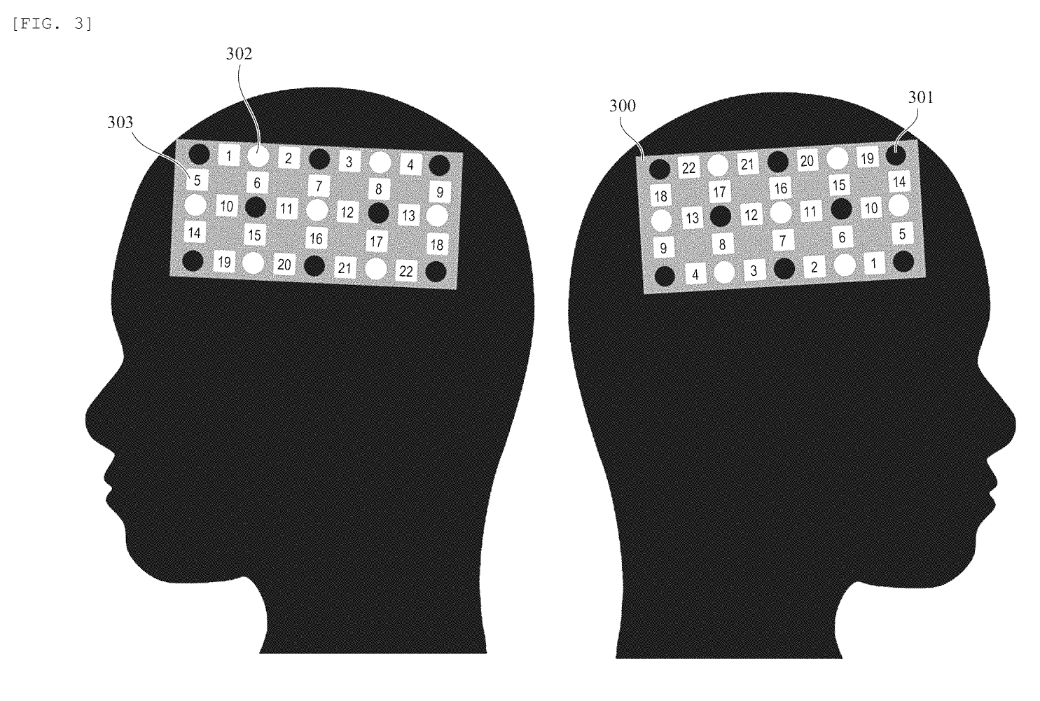

FIG. 3 is a diagram representing an example of the way that a measurement probe 300 is fitted to the head of a subject (patient) for the measurement of biological signals (brain signals) from patient.

The measurement probe 300 is configured from an arrangement of plural optical sources 301, plural detectors 302, and plural measurement point channels 303. The placement of measurement probe 300 depends on a hypothesis for a cognitive task performed by a patient. The measurement probe 300 is placed, for example, at the frontal lobe and the frontal-parietal region in the case of an inhibition task and an attention task, and at the prefrontal cortex (PFC) region in the case of a working memory task.

### Data Sequence

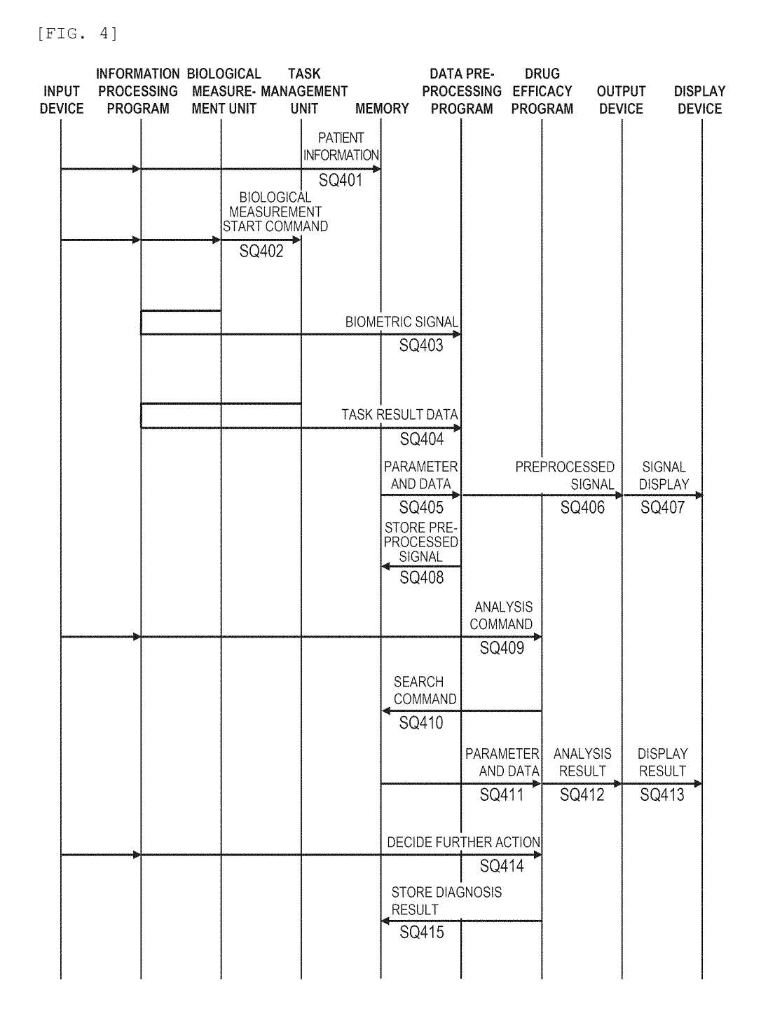

FIG. 4 is a diagram representing a data sequence in the drug efficacy evaluation assisting system 1 of the present embodiment. In the present embodiment, all inputs are received by the information processing program 103, and the information processing program 103 determines whether the input data or information should be transferred to which unit or program before the data or information are input to the intended unit or program. However, data or information may be directly input to the intended unit or program.

### (i) Sequence 401

Before biological signal measurement of a patient, a doctor or an operator enters personal information of the patient using the input device 101. In response, the information processing program 103 stores the patient's personal information in the private database 109a of the memory 109.

### (ii) Sequence 402

In order to start a biological signal measurement, a doctor or an operator enters a biological measurement start command using the input device 101. In response, the information processing program 103 transfers the biological measurement start command to the biological measurement unit 106 and the task management unit 107.

(iii) Sequence 403

The biological measurement unit 106 sends the measured biological signal (biometric signal: for example, an optical brain-function measurement signal, an NIRS signal, or a brain signal) to the data preprocessing program 104 via the information processing program 103.

### (iv) Sequence 404

As with the case of the biometric signal, the task management unit 107 sends the performed task result data of the patient to the data preprocessing program 104 via the information processing program 103.

### (v) Sequence 405

The data preprocessing program 104 acquires an analysis parameter and data from the analysis parameter database 109c of the memory 109, and preprocesses these data. Specifically, the data preprocessing program 104 reads out the stored data in the analysis parameter database 109c of the memory 109, including, for example, data from the motion elimination 216 to the subject of noise correction 220, and removes motion and noise from the biometric signal acquired from the biological measurement unit 106 (corresponding to a preprocess), and generates a preprocessed biometric signal.

### (vi) Sequence 406

The data preprocessing program 104 sends the preprocessed biometric signal (biometric signal after noise removal) to the output device 110.

(vii) Sequence 407

The output device 110 processes the biometric signal according to the display conditions of the display device, and sends the processed signal to the display device 108. In response, the display device 108 displays the biometric signal on the screen, enabling the doctor or operator to visually confirm the biometric signal.

(viii) Sequence 408

The data preprocessing program 104 stores the preprocessed biometric signal in the measurement database 109b of the memory 109. This biometric signal is used for further processes.

### (ix) Sequence 409

When there is a need to analyze the biometric signal further, the doctor or operator enters an analysis command using the input device 101. The entered analysis command is received by the drug efficacy program 105 via the information processing program 103.

### (x) Sequence 410

The current biometric signal, and/or the data stored in the measurement database 109b are used for efficacy analysis and response prediction analysis. To this end, the drug efficacy program 105 sends a search command for the required biometric signal and/or data to the memory 109.

### (xi) Sequence 411

From the memory 109, the drug efficacy program 105 acquires a biometric signal and/or data corresponding to the search command, and executes an efficacy analysis or a response prediction analysis.

(xii) Sequence 412

The drug efficacy program 105 sends the analysis result to the output device 110. The output device 110 executes a predetermined process on the analysis result data according to the display conditions of the display device 108.

(xiii) Sequence 413

The output device 110 sends the analysis result data to the display device 108, and the display device 108 displays the received analysis result data on the screen. The analysis result includes the diagnosis result (the presence or absence of efficacy) automatically determined by the drug efficacy program 105.

(xiv) Sequence 414

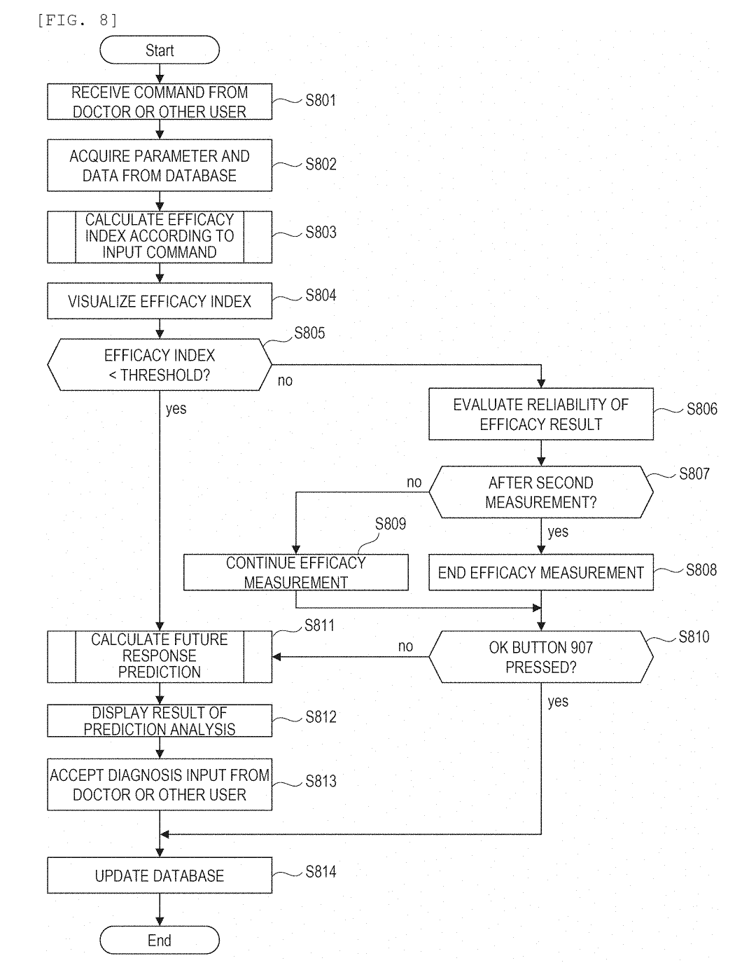

The doctor or operator decides a further action (end efficacy measurement, continue efficacy measurement, or predict response; see FIG. 9) from the diagnosis result automatically determined by the drug efficacy program 105. In response to the doctor or operator entering the decided further action using the input device 101, the drug efficacy program 105 acquires the decided further action via the information processing program 103.

### (xv) Sequence 415

The doctor or operator presses the OK button (see FIG. 9) when he or she agrees with the diagnosis result automatically determined by the drug efficacy program 105. In response, the drug efficacy program 105 stores the diagnosis result in the measurement database 109b of the memory 109. When the diagnosis result automatically determined by the drug efficacy program 105 is not acceptable, the doctor or operator may enter the diagnosis result that has been determined by the doctor himself or herself or by some other person from the analysis result, using the input device 101, and the diagnosis result may be stored in the measurement database 109b.

### Exemplary Structure of Channel Selecting Display Screen

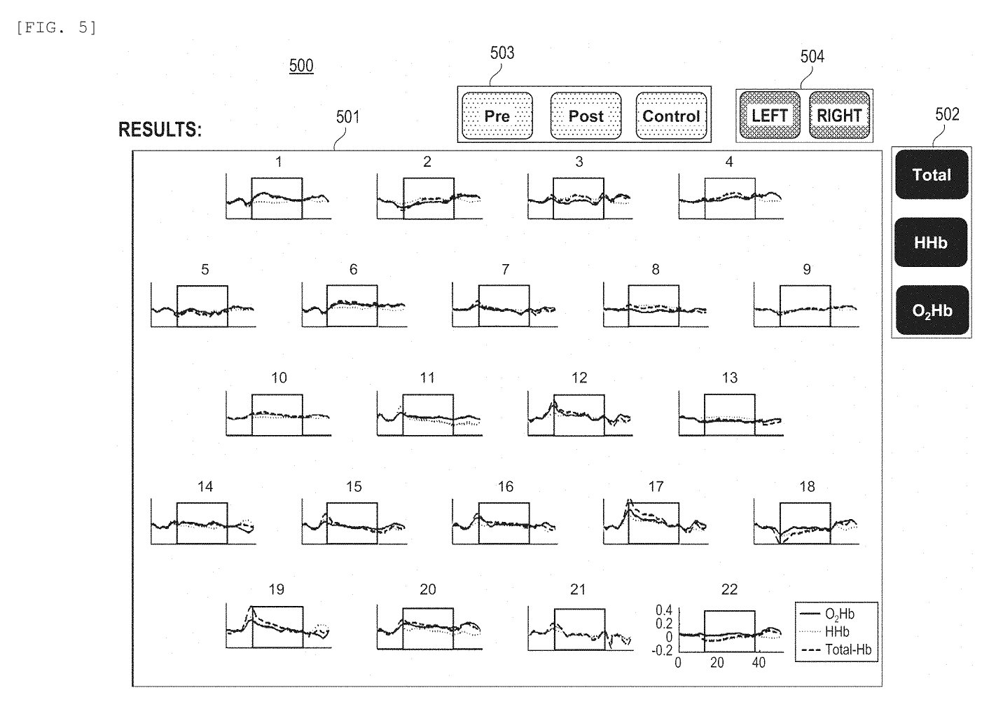

FIG. 5 is a diagram showing an exemplary structure of a channel selecting display screen 500 of the present embodiment. A channel to be displayed in detail (see FIG. 6) can be selected by using the selecting screen (GUI) 500.

The channel selecting display screen 500 is configured to display items that includes a biometric signal display region 501, hemoglobin type selecting button 502, a measurement status selecting button 503, and a cerebral hemisphere selecting button 504.

The number of biometric signals displayed on the channel selecting display screen 500 is equal to the number of measurement points or channels in the measurement probe 300.

The biometric signal display region 501 is an overall display of the biometric signal of each channel on the measurement probe. Upon a doctor or other user selecting one of the biometric signals, the biometric signal selected from the overall display can be displayed in detail. The biometric signal (preprocessed) displayed in the biometric signal display region 501 includes changes in oxygenated hemoglobin concentration (O₂Hb), deoxygenated hemoglobin concentration (HHb), and total hemoglobin concentration (Total) over time at each channel. These hemoglobin concentration changes reflect brain activity.

The hemoglobin type selecting button 502 is used to select which of the oxygenated hemoglobin concentration (O₂Hb), deoxygenated hemoglobin concentration (HHb), and total hemoglobin concentration (Total) changes over time is to be displayed in detail.

The measurement status selecting button 503 is used to select which of the patient's biometric signal before administration (Pre), the patient's biometric signal after administration (Post), and the biometric signal of a healthy individual (Control) is to be displayed in detail.

The cerebral hemisphere selecting button 504 is used to select the right brain or left brain.

With the channel selecting display screen 500, a doctor or other user can predict the result of automatic analysis from experience by looking at the whole view of the acquired biometric signals. By selecting a channel which is desired to look at in detail, a doctor also can examine the signal of interest in detail.

### Examples of Detailed Biometric Signal Display

FIG. 6 is a diagram showing details of hemoglobin waveform 600 at the selected channel before and after drug administration.

The details of hemoglobin waveform 600 before and after administration include display items that includes a selected channel 601 indicative of the selected channel, a selected signal display region 602 indicative of the selected biometric signal, and an activity interval (stimulus period) 603 representing an interval in which a part of the patient's response signal (biometric signal) to a given task is extracted for analysis.

The display shown in FIG. 6 is an example in which the biometric signal is of a channel 10, the hemoglobin type is O₂Hb, the measurement status is all of Pre, Post, and Control, and the cerebral hemisphere is the right brain after the selection in FIG. 5.

By looking at the detailed display, a doctor or other user can compare a brain activity change at a specific location (channel) of the patient before and after drug administration, or brain activity differences between a healthy individual and the patient.

### Exemplary Structure of Command Input GUI

FIG. 7 is a diagram showing an exemplary structure of a GUI 700 that a doctor or an operator uses to enter an analysis-prediction command.

The GUI 700 for entering an analysis-prediction command is configured to display items that include a selection of efficacy indices (Efficacy index) 701, a selection of activity intervals 702, a selection of prediction methods 703, a selection of option variables 704, a reset button 705, and an OK button 706.

The selection of efficacy indices 701 is used to select which index is used for the analysis of the presence or absence of efficacy, and allows a doctor or other user to select, for example, Hb change (simple modulation (modulation)), Hb change (z-score), or activity-variability (Activity-Variability). When selecting Hb change (simple modulation (modulation)) or Hb change (z-score), the activity interval can be selected using the selection of activity intervals 702. When “Auto” (default) in the upper field of the selection of activity intervals 702 is selected, the activity interval 222 is used upon reading it from the analysis parameter database 109c. When “Specify” is selected in the upper field of the selection of activity intervals 702, an interval can be specified in the lower field of the selection of activity intervals 702. For analysis of a biometric signal, a part of the measured biometric signals of all patients (population data) corresponding to the set activity interval is extracted, and used for analysis.

The selection of prediction methods 703 is used to select a method used to predict the process result after analysis, and allows a doctor or other user to select, for example, dose-index relation (Dose-index relation), sigmoid function fitting (Sigmoid), and multiple linear regression (Multiple linear regression).

The selection of option variables 704 is used to specify a variable to be used in multiple linear regression, and the variable can be selected from, for example, dose, age, and gender.

The reset button 705 is used to reset the set command. The OK button 706 is used to apply the set command.

### Overview of Processes by Drug Efficacy Program

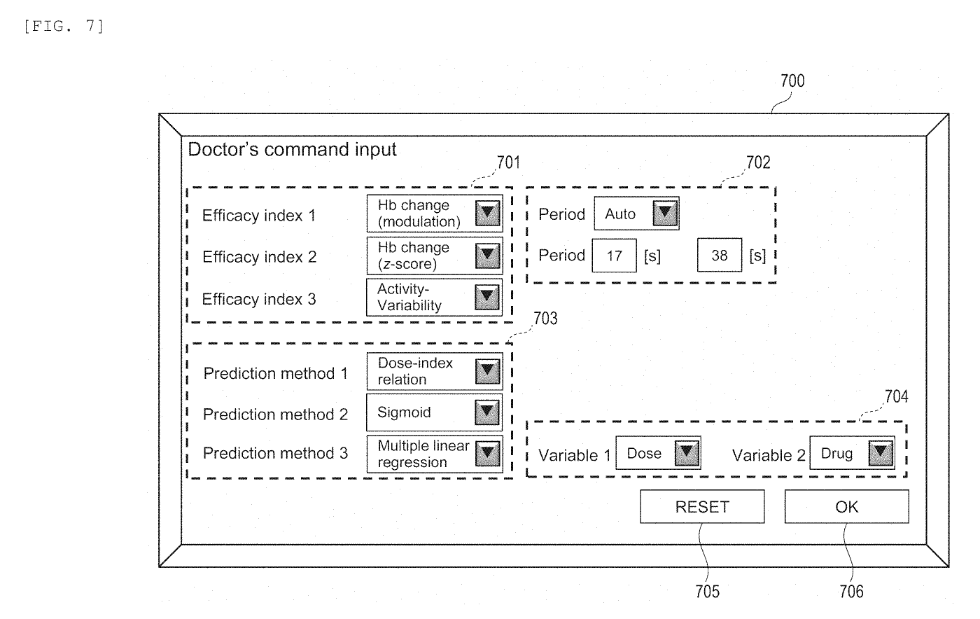

FIG. 8 is a flowchart explaining an overview of the processes by the drug efficacy program 105 of the present embodiment.

### (i) Step 801

Upon a doctor or other user pressing the OK button 706 in the analysis-prediction command input GUI 700 (FIG. 7), the drug efficacy program 105 accepts the commands set in the analysis-prediction command input GUI 700.

### (ii) Step 802

The drug efficacy program 105 acquires the analysis parameter and other data from the databases of the memory 109, as required.

(iii) Step 803

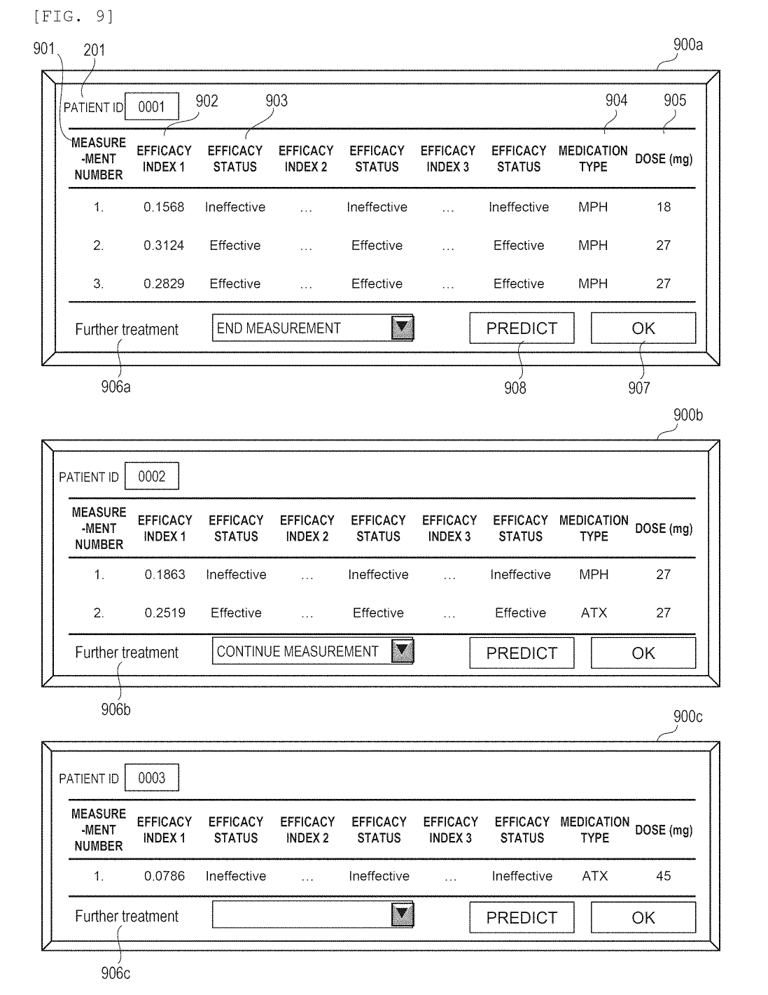

To the data acquired in step 802, the drug efficacy index/coefficient program 105a applies the analysis method specified by the analysis command (see FIG. 7), and calculates an efficacy index for the patient being measured. Step 803 will be described in detail below with reference to FIG. 10 and other figures.

### (iv) Step 804

The drug efficacy program 105 visualizes (displays) the efficacy index calculated in step 803.

### (v) Step 805

The drug efficacy program 105 compares the threshold drawn from the efficacy index calculated in step 803 (the threshold depends on the analysis technique) with the efficacy index obtained from the current biometric signal of the patient, and determines whether the current biometric signal is below the threshold. The sequence goes to step 811 when the current efficacy index data is below the threshold (Yes in step 805). When the current efficacy index data is equal to or greater than the threshold (No in step 805), the sequence goes to step 806.

### (vi) Step 806

The drug efficacy program 105 evaluates the reliability of the efficacy result by referring to the past measurement results, with regard to whether administration of the same drug in the same amount has produced efficacy. In the present embodiment, the program assesses, for example, the number of times the biological signal measurements have been performed. However, assessment may be made for other items (for example, the rating scale of the patient of interest, task correctness, and the extent of the deviation of the current efficacy index data from the threshold).

(vii) Step 807

The drug efficacy program 105 determines whether the measurement count is larger than 2. The sequence goes to step 808 when the measurement count is larger than 2 (Yes in step 807). When the measurement count is 2 or less (No in step 807), the sequence goes to step 809.

(viii) Step 808

The drug efficacy program 105 proposes ending the efficacy measurement (End efficacy measurement). Here, ending of efficacy measurement is a default setting. A doctor or other user can change the setting, as desired.

### (ix) Step 809

The drug efficacy program 105 proposes continuing the efficacy measurement. The measurement is repeated at a later date under the same conditions, and the efficacy level is reevaluated. Here, continuing of efficacy measurement (Continue efficacy measurement) is a default setting. A doctor or other user can change the setting, as desired.

### (x) Step 810

The drug efficacy program 105 determines whether a doctor or other user has pressed the OK button 907 (see FIG. 9), or the prediction (Prediction) button 908. The sequence goes to step 814 when the OK button 907 is pressed (Yes in step 810). When the prediction button 908 is pressed (No in step 810), the sequence goes to step 811.

### (xi) Step 811

To the data acquired in step 802, the response prediction program 105b applies the prediction (analysis) method specified by the set prediction command (see FIG. 7), and predicts (analyzes) a future response of the patient of interest. Step 811 will be described in detail below with reference to FIG. 18 and other figures.

(xii) Step 812

The drug efficacy program 105 displays the results of prediction and analysis on the screen of the display device 108. Visualizing the prediction and analysis results enables a doctor or other user, including a caregiver of a patient, to readily understand and determine how to proceed with the treatment.

(xiii) Step 813

The drug efficacy program 105 accepts a diagnosis input from a doctor or other user, if the doctor or the user has entered a diagnosis by himself or herself.

(xiv) Step 814

The drug efficacy program 105 stores the content of the input diagnosis in the diagnosis result 214 of the measurement database 109b. When the OK button 907 is pressed in step 810, the drug efficacy program 105 determines that “Effective” was approved by a doctor or other user, and stores the effective diagnosis (default value) in the diagnosis result 214 of the measurement database 109b.

### List Display of Efficacy Analysis Result (Examples)

FIG. 9 is a diagram showing examples of a list display of the efficacy analysis result of the present embodiment. The efficacy analysis result list displays 900a to 900c are configured to display items that include a patient ID 201, a measurement count 901, an index 902 indicative of the calculated index, an efficacy status 903 indicative of the diagnosis result concerning the efficacy of the corresponding index, a medication type 904, a dose 905, a further treatment (Further treatment) 906a to 906c, an OK button 907 used to end measurement, and a prediction button 908 used to start prediction.

As shown in FIG. 9, the efficacy analysis result is displayed as a list in three different forms that vary with the patient's conditions. The efficacy index 902 is calculated for the data of all measurements according to the entered efficacy index (analysis) command (701 in FIG. 7). As described above, the index calculated for the patient of interest is compared with the calculated threshold, and the efficacy status 903 is evaluated. A further treatment (Further treatment) is automatically presented to a doctor or other user according to the measurement count 901, and the efficacy analysis result (efficacy status 903).

The displayed contents related to further treatment include “End measurement” 906a (the efficacy is sufficient), “Continue measurement” 906b (the reliability is not sufficient at this stage), and blank 906c (efficacy is determined to be absent). The blank 906c suggests further execution of a prediction analysis (Prediction) to a doctor or other user. In this case, a doctor or other user may press the prediction button 908 to execute a prediction analysis, and make a plan for further treatment. The doctor or other user presses the OK button 907 when he or she approves “End measurement” 906a or “Continue measurement” 906b.

### Details of Computation of Efficacy Index (Step 803)

FIGS. 10A and 10B are flowcharts explaining details of the computation of efficacy index (step 803) of the present embodiment. The method of calculation of efficacy index depends on the selected analysis method (701 in FIG. 7).

### (i) Step 1001

The drug efficacy index/coefficient program 105a reads the command entered by a doctor or other user (the efficacy index set in the selection of efficacy indices 701). In the present embodiment, the process is performed in series for each command when more than one efficacy index is set. However, the process may be performed in parallel.

### (ii) Step 1002

The drug efficacy index/coefficient program 105a determines whether the command is “Hb change”. The sequence goes to step 1004 when the command is “Hb change” (Yes in step 1002). When the command is not “Hb change” (No in step 1002), the sequence goes to step 1003.

(iii) Step 1003

The drug efficacy index/coefficient program 105a executes an activity-variability (Activity-Variability) analysis. Step 1003 will be described in detail below with reference to FIG. 11 and other figures.

### (iv) Step 1004

The drug efficacy index/coefficient program 105a determines whether the command set for activity interval is “Auto”. The sequence goes to step 1005 when the command set for activity interval is “Auto” (Yes in step 1004). When the command set for activity interval is not “Auto” (No in step 1004), the sequence goes to step 1006.

### (v) Step 1005

The drug efficacy index/coefficient program 105a reads the value of activity interval 222 from the analysis parameter database 109c.

### (vi) Step 1006

The drug efficacy index/coefficient program 105a reads the value of the activity interval set by a doctor or other user.

(vii) Step 1007

The drug efficacy index/coefficient program 105a refers to the measurement database 109b, and recognizes the type of performed task, and the type of administered drug in the current measurement of the patient of interest.

(viii) Step 1008

Concerning the same task and the same administered drug as those specified in step 1007, the drug efficacy index/coefficient program 105a acquires the patient data (biometric signal) before and after administration (Pre and Post), or an extracted signal 210 from the measurement database 109b.

### (ix) Step 1009

For the data acquired in step 1008, the drug efficacy index/coefficient program 105a calculates the average Hb change in the given activity interval.

### (x) Step 1010

The drug efficacy index/coefficient program 105a determines whether the command is Hb change (simple modulation). The sequence goes to step 1011 when the command is Hb change (simple modulation) (Yes in step 1010). When the command is not Hb change (simple modulation) (No in step 1010), the sequence goes to step 1016.

### (xi) Step 1011

For all patients stored in the measurement database 109b, the drug efficacy index/coefficient program 105a subtracts pre-administration data (Pre) from post-administration data (Post) (see formula 1) to calculate a neuromodulation index.

$$\begin{matrix}[ Math . 1 ] & \\ Index ( i ) = \frac{{\sum }_{t = t 1}^{t 2} Δ {C ( t )}_{{( Hb )}_{post}}}{t 2 - t 1} - \frac{{\sum }_{t = t 1}^{t 2} Δ {C ( t )}_{{( Hb )}_{pre}}}{t 2 - t 1} & ( Formula 1 )\end{matrix}$$

Here, i represents the position in the order of patients in a patient group before or after administration, t1 represents the start time of the calculation of hemoglobin concentration change, t2 represents the end time of the calculation of hemoglobin concentration change, ΔC_{(Hb)} represents the hemoglobin concentration change, pre represents the state before administration, and post represents the state after administration.

(xii) Step 1012

The drug efficacy index/coefficient program 105a calculates a normal distribution of neuromodulation index. The distribution becomes closer to a true distribution, and the reliability improves when larger data volumes from larger numbers of patients are used.

(xiii) Step 1013

The drug efficacy index/coefficient program 105a determines the threshold, taking into account parameters of distribution characteristics in step 1012 (for example, the mean value, the standard deviation, and the distribution type). As an example, the threshold may be a mean value.

(xiv) Step 1014

The drug efficacy index/coefficient program 105a plots the normal distribution calculated in step 1012.

### (xv) Step 1015

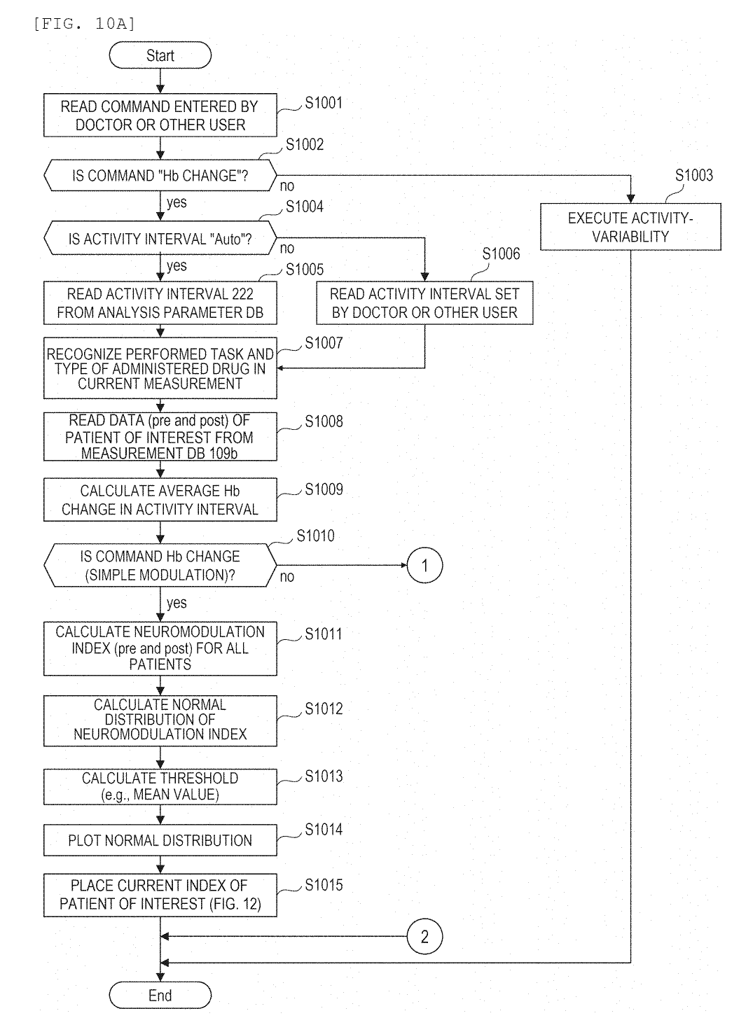

The drug efficacy index/coefficient program 105a places the current index of the patient of interest on the normal distribution (see FIG. 12).

(xvi) Step 1016

The drug efficacy index/coefficient program 105a calculates a normal distribution for different conditions of all patient data (before and after administration (Pre and Post)).

(xvii) Step 1017

The drug efficacy index/coefficient program 105a calculates z-scores (z-score before administration, and z-score after administration) for the data of all patients according to the following formulae 2 to 5.

$$\begin{matrix}[ Math . 2 ] & \\ {avg ( i )}_{pre / post} = \frac{1}{t 2 - t 1} {\sum }_{t = t 1}^{t 2} Δ {C ( t )}_{{( Hb )}_{pre / post}} & ( Formula 2 )\end{matrix}$$

Here, i represents the position in the order of patients in a patient group before or after administration (patient index), avg represents the hemoglobin concentration change (ΔC_{(Hb)}: average value of brain activity in an activity interval from t=t1 to t=t2), pre represents the state before administration, and post represents the state after administration.

$$\begin{matrix}[ Math . 3 ] & \\ {μ}_{pre / post} = \frac{1}{n} {\sum }_{i = 1}^{n} {avg ( i )}_{pre / post} & ( Formula 3 ) \\ {σ}_{pre / post} = \sqrt{\frac{1}{n} {\sum }_{i = 1}^{n} {( {avg ( i )}_{pre / post} - {μ}_{pre / post} )}^{2}} & ( Formula 4 ) \\ {z ( i )}_{pre / post} = \frac{{avg ( i )}_{pre / post} - {μ}_{pre / post}}{{σ}_{pre / post}} & ( Formula 5 )\end{matrix}$$

Here, μ represents the mean value of brain activity in patients in a patient group before and after administration, σ represents the standard deviation of brain activity in patients in a patient group before and after administration, n represents the total number of patients in a group before administration and in a group after administration, and the z-score represents the standardized value of brain activity in a patient group before and after administration.

(xviii) Step 1018

The drug efficacy index/coefficient program 105a determines whether the command is Hb change (z-score). The sequence goes to step 1019 when the command is Hb change (z-score) (Yes in step 1018). When the command is not Hb change (z-score) (No in step 1018), the sequence goes to step 1025.

(xix) Step 1019

The drug efficacy index/coefficient program 105a calculates a z-score for simulation data (data assuming that the average brain activity (Hb concentration) data before administration (Pre) and the average brain activity (Hb concentration) data after administration (Post) are the same) according to formulae 6 to 8.

$$\begin{matrix}[ Math . 4 ] & \\ avg = {avg}_{pre} = {avg}_{post} & ( Formula 6 ) \\ {z}_{pre \_ sim} = \frac{avg - {μ}_{pre}}{{σ}_{pre}} & ( Formula 7 ) \\ {z}_{post \_ sim} = \frac{avg - {μ}_{post}}{{σ}_{post}} & ( Formula 8 )\end{matrix}$$

Here, avg represents simulation data for a patient group before administration (zₚᵣₑ_{_}ₛᵢₘ), and for a patient group after administration (zₚₒₛₜ_{_}ₛᵢₘ).

### (xx) Step 1020

For the simulation data, the drug efficacy index/coefficient program 105a sets the pre-administration z-score on X axis, and the post-administration z-score on Y axis, and calculates a linear regression line (refer to Formulae 9 and 10).

[Math. 5]

threshold(1401)=(zₚᵣₑ_{_}ₛᵢₘ,zₚₒₛₜ_{_}ₛᵢₘ) (Formula 9)

zₚₒₛₜ_{_}ₛᵢₘ=zₚᵣₑ_{_}ₛᵢₘβ+C (Formula 10)

The threshold is represented by the linear regression line. Here, β and C are coefficients of the linear regression line.
(xxi) Step 1021

For all patient data, the drug efficacy index/coefficient program 105a pairs the pre-administration z-score (X axis) and the post-administration z-score (Y axis), and calculates the distance from each patient data to the linear regression line determined in step 1020, according to formula

$$\begin{matrix}[ Math . 6 ] & \\ dist ( i ) = \frac{\vert {z ( i )}_{post} - {z ( i )}_{pre} β - C \vert}{\sqrt{{1}^{2} + {β}^{2}}} & ( Formula 11 )\end{matrix}$$

Here, dist represents the distance between the values (z(i)ₚᵣₑ, and z(i)ₚₒₛₜ) of individual patients and the threshold line (linear regression line). The data have positive modulation when avg(i)ₚₒₛₜ>avg(i)ₚᵣₑ, and negative modulation when avg(i)ₚₒₛₜ<avg(i)ₚᵣₑ.

(xxii) Step 1022

The drug efficacy index/coefficient program 105a distinguishes two regions. Specifically, the region above the linear regression line is a positive modulation region, and the region below the linear regression line is a negative modulation region. For each region, the drug efficacy index/coefficient program 105a calculates the average (average distance) of the distances from each point to the linear regression line.

$$\begin{matrix}[ Math . 7 ] & \\ {μ}_{{dist}_{pos / neg}} = \frac{1}{n} {\sum }_{i = 1}^{n} {dist ( i )}_{pos / neg} & ( Formula 12 )\end{matrix}$$

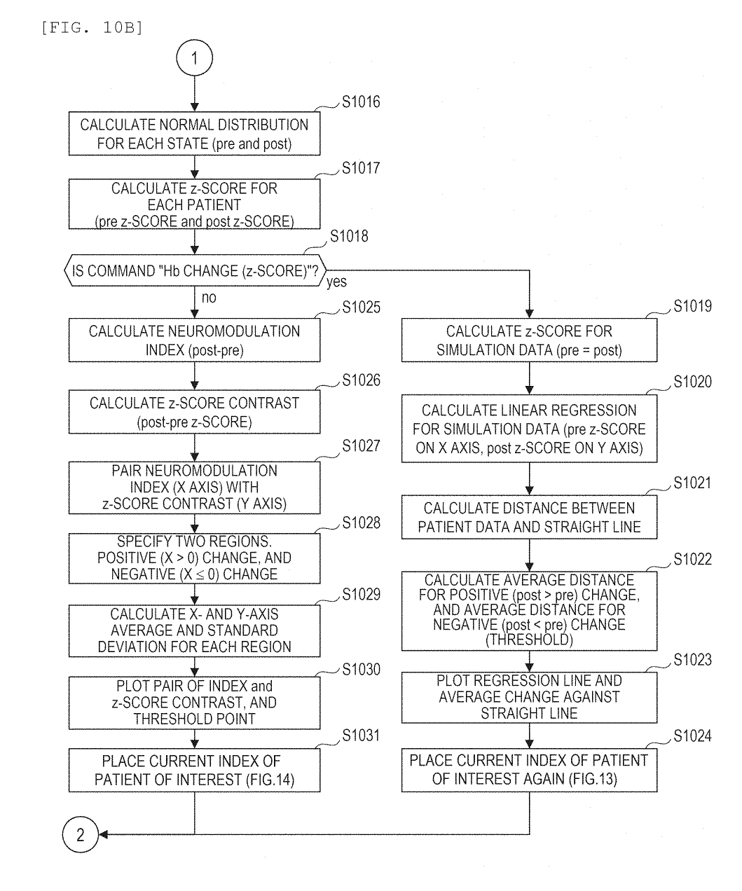

Here, μ_{dist} represents the average distance from the measured patient's data to the regression line 1301 separating positive modulation and negative modulation (see FIG. 13).

(xxiii) Step 1023

The drug efficacy index/coefficient program 105a plots a linear regression line and an average distance line.

(xxiv) Step 1024

The drug efficacy index/coefficient program 105a places the current index of the patient of interest (see FIG. 13).

(xxv) Step 1025

For all patients stored in the measurement database 109b, the drug efficacy index/coefficient program 105a subtracts the pre-administration data (Pre) from the post-administration data (Post), and calculates the neuromodulation index according to the formula 1 above.

(xxvi) Step 1026

The drug efficacy index/coefficient program 105a calculates the contrast between the z-score of average brain activity before administration, and the z-score of average brain activity after administration (z-score contrast: Z_{Post}−Z_{Pre}) according to formulae 2 to 5, and formula 13.

[Math. 8]

z(i)_{contrast}=z(i)ₚₒₛₜ−Z(i)ₚᵣₑ (Formula 13)

Here, z(i)_{contrast} represents the contrast between z-scores, specifically the difference between the standardized brain activity before administration and the standardized brain activity after administration.

(xxvii) Step 1027

The drug efficacy index/coefficient program 105a places the neuromodulation index on X axis, and the z-score contrast on Y axis, and pairs these index and contrast.

(xxviii) Step 1028

The drug efficacy index/coefficient program 105a distinguishes two regions: a region with reduced brain activity after administration (X<0; negative modulation), and a region with increased brain activity after administration (X>0; positive modulation), relative to the threshold X=0. At X=0, there is no change in brain activity (Hb concentration) before and after administration.

(xxix) Step 1029

For each region, the drug efficacy index/coefficient program 105a calculates a data distribution of X-axis and Y-axis data (mean and standard deviation).

(xxx) Step 1030

The drug efficacy index/coefficient program 105a plots the pair of neuromodulation index and z-score contrast, along with the threshold.

(xxxi) Step 1031

The drug efficacy index/coefficient program 105a places the current index of the patient of interest (see FIG. 14). The personal index of the patient is placed at the coordinates (index(i), z(i)_{contrast}). The data has positive modulation when index>0, and negative modulation when index<0.

### Details of Efficacy Index Computation by Activity-Variability Analysis

FIG. 11 is a flowchart explaining details of the efficacy index computation by activity-variability analysis (step 1003).

### (i) Step 1101

The drug efficacy index/coefficient program 105a reads out the value of activity interval 222 (Auto) from the analysis parameter database 109c.

### (ii) Step 1102

The drug efficacy index/coefficient program 105a refers to the measurement database 109b, and recognizes the type of performed task, and the type of administered drug in the current measurement of the patient of interest.

(iii) Step 1103

From the measurement database 109b, the drug efficacy index/coefficient program 105a reads the data of all healthy individuals, and the data of all patients who have undertaken the same task and had the same drug as the patient of interest (pre-administration data, and post-administration data).

### (iv) Step 1104

The drug efficacy index/coefficient program 105a calculates the average of Hb changes (brain activity) in the activity interval, according to formula 14.

$$\begin{matrix} [ Math . 9 ] & \\ {avg ( i )}_{pre / post / control} = \frac{1}{t 2 - t 1} {\sum }_{t = t 1}^{t 2} ( \frac{1}{m} {\sum }_{tr = 1}^{m} Δ {C ( i , tr , t )}_{{( Hb )}_{pre / post / control}} ) & ( Formula 14 )\end{matrix}$$

Here, i represents the position in the order of patients or healthy individuals in a patient group before administration, a patient group after administration, and a healthy individual group, t1 represents the start time of the calculation of hemoglobin concentration change, t2 represents the end time of the calculation of hemoglobin concentration change, tr represents the number of the task trials performed in a single measurement, pre represents the state before administration, post represents the state after administration, control represents the state of a healthy individual, and ΔC_{(Hb)} represents the hemoglobin concentration change.

### (v) Step 1105

A patient may be asked to undertake more than one task trial in a single measurement. For such a situation, the drug efficacy index/coefficient program 105a calculates a statistical dispersion variable (for example, standard deviation, variance, or variability) for multiple task trials according to formula 15.

$$\begin{matrix}[ Math . 10 ] & \\ {σ ( i )}_{pre / post / control} = \sqrt{\frac{1}{m} {\sum }_{tr = 1}^{m} {( \begin{matrix}[ \frac{1}{t 2 - t 1} {\sum }_{t = t 1}^{t 2} Δ {C ( i , tr , t )}_{{( Hb )}_{pre / post / control}} ] - \\ {avg ( i )}_{pre / post / control}\end{matrix} )}^{2}} & ( Formula 15 )\end{matrix}$$

Here, σ represents the standard deviation of each individual (a patient before and after administration, a healthy individual) undertaking task trials.

### (vi) Step 1106

The drug efficacy index/coefficient program 105a sets the mean value of Hb change on X axis, and the statistical dispersion variable on Y axis.

(vii) Step 1107

The drug efficacy index/coefficient program 105a separates the data into two regions using a predetermined clustering method (for example, k-means clustering).

(viii) Step 1108

The drug efficacy index/coefficient program 105a acquires a probability index, and center distribution data for the data of each region obtained in step 1107.

### (ix) Step 1109

The line separating the two regions is determined as the threshold line by the drug efficacy index/coefficient program 105a.

### (x) Step 1110

The drug efficacy index/coefficient program 105a confirms the sensitivity and the specificity of the clustering result. Here, the regions are based on the data of healthy individuals, and as such the drug efficacy index/coefficient program 105a checks a distribution of individuals (patients, healthy patients) in each region.

### (xi) Step 1111

The drug efficacy index/coefficient program 105a selects a region with more healthy individuals as an efficacy modulated cluster (a cluster with modulation due to efficacy).

(xii) Step 1112

The drug efficacy index/coefficient program 105a plots the threshold line and the center of the cluster.

(xiii) Step 1113

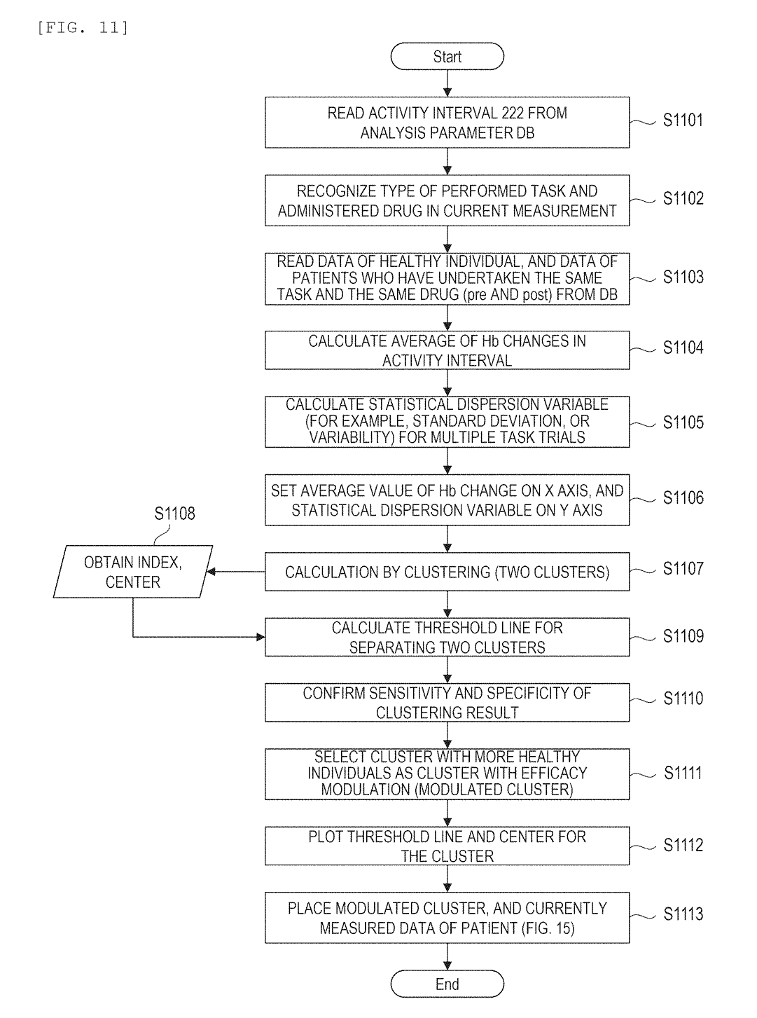

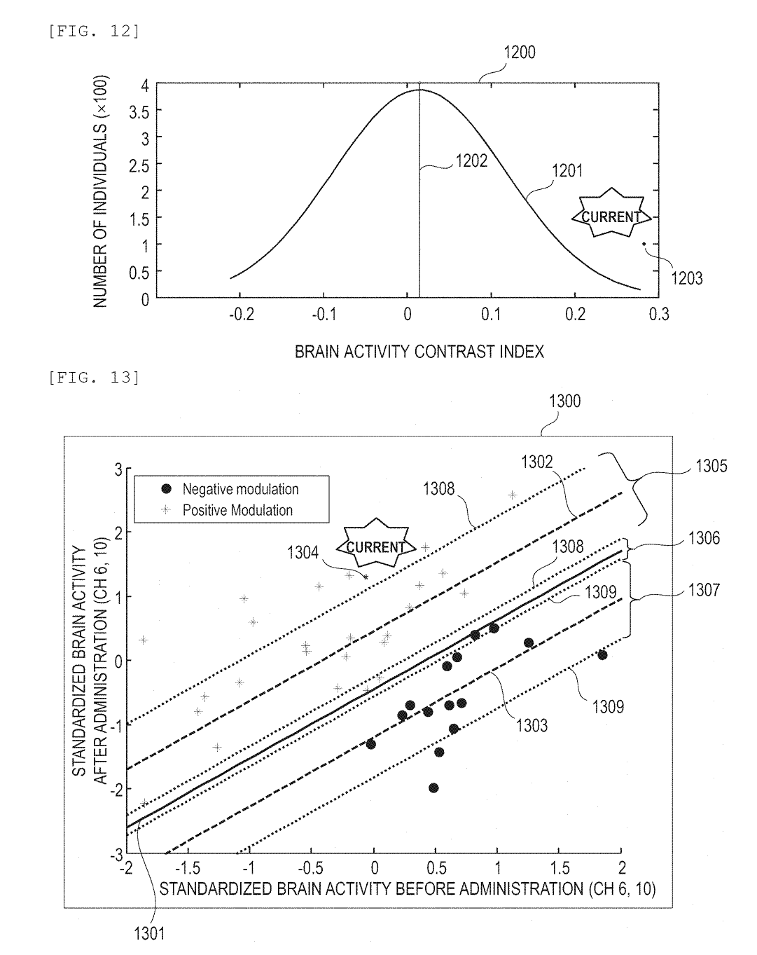

The drug efficacy index/coefficient program 105a places the efficacy modulated cluster, and the current measurement result of the patient of interest (see FIG. 15).

### Graph of Efficacy Index of Hb Change (Simple Modulation) (Example)

FIG. 12 is a graph 1200 representing the efficacy index of Hb change (simple modulation) (a diagram representing efficacy for the patient (subject) of interest against a comparison group).

The graph 1200 represents a normal distribution of the neuromodulation indices obtained from all patients who have undertaken the same task, and had the same drug. A normal distribution curve 1201 is plotted in the graph 1200. The mean value of neuromodulation indices (the index value that takes the maximum) is set as threshold 1202.

As shown in FIG. 12, the index value 1203 of the current measurement is placed in the graph 1200. Here, the index value is larger than the threshold 1202, and can be determined as having efficacy.

Scatter Chart Sorting Efficacy by Hb Change (z-Score) (Example)

FIG. 13 shows a scatter chart 1300 sorting efficacy by Hb change (z-score). The scatter chart 1300 visualizes the standardized value (z-score) of the result before administration, and the standardized value (z-score) after administration by marking these on X axis and Y axis, respectively.

The graph is separated into two regions by the regression line 1301. The upper region represents positive modulation, and the lower region represents negative modulation. The distance from each data to the regression line 1301, and the mean value of distances in each region are calculated.

A mean distance line 1302 and a mean distance line 1303 are plotted in the positive modulation region and the negative modulation region, respectively. The current measurement result (z-score based on pre-administration data and post-administration data) 1304 is also placed (specified) in the graph. It can be seen that the current measurement result 1304 falls in the positive modulation region including the effective region 1305.

The standard deviation in each region is plotted as dotted lines 1308 and 1309, according to formula 16.

$$\begin{matrix}[ Math . 11 ] & \\ {σ}_{{dist}_{pos} / neg} = \sqrt{\frac{1}{n} {\sum }_{i = 1}^{n} {( {dist ( i )}_{pos / neg} - {μ}_{{dist}_{pos} / neg} )}^{2}} & ( Formula 16 )\end{matrix}$$

Here, σ represents the standard deviation of the distance to the regression line 1301 in the positive modulation group and the negative modulation group.

The region 1306 confined between the two standard deviation dotted lines 1308 and 1309 in the vicinity of the regression line 1301 represents a region where efficacy cannot be definitively determined (low efficacy, or efficacy is small, if any). It is accordingly desirable to repeat the measurement when the measurement result falls in the region 1306.

As can be understood from the foregoing descriptions, the region 1305 is a region where the presence of efficacy can be definitively determined, whereas the region 1307 is a region where efficacy can be determined as being absent.

Scatter Chart Sorting Efficacy by Hb Change (z-Score Contrast) (Example)

FIG. 14 shows a scatter chart 1400 sorting efficacy by Hb change (z-score contrast).

In the scatter chart 1400, neuromodulation index and z-score contrast (zₚₒₛₜ−zₚᵣₑ) are set on X axis and Y axis, respectively. The measurement results are placed in the vicinity of the straight line 1401. X-axis values (neuromodulation index values) smaller than 0 mean that the brain activity decreased after drug administration (negative modulation), whereas X-axis values (neuromodulation index values) larger than 0 mean that the brain activity increased after drug administration (positive modulation).

Calculations are performed to find a center 1402 in the positive modulation region, and a center 1403 in the negative modulation region, according to formula 17. These are placed on the scatter chart 1400.

$$\begin{matrix} [ Math . 12 ] & \\ {Center}_{pos / neg} ( 1402 / 1403 ) = ( \frac{1}{n} {\sum }_{i = 1}^{n} {Index ( i )}_{pos / neg} , \frac{1}{n} {\sum }_{i = 1}^{n} {z ( i )}_{{contrast}_{pos / neg}} ) & ( Formula 17 ) \\ {d}_{pos / neg} = \sqrt{\begin{matrix}{( {Index ( i )}_{pos / neg} - \frac{1}{n} {\sum }_{i = 1}^{n} {Index ( i )}_{pos / neg} )}^{2} + \\ {( {z ( i )}_{{contrast}_{pos / neg}} - \frac{1}{n} {\sum }_{i = 1}^{n} {z ( i )}_{{contrast}_{pos / neg}} )}^{2}\end{matrix}} & ( Formula 18 ) \\ {μ}_{{d}_{pos / neg }} = \frac{1}{n} {\sum }_{i = 1}^{n} {d ( i )}_{pos / neg} & ( Formula 19 ) \\ {σ}_{{d}_{pos / neg}} = \sqrt{\frac{1}{n} {\sum }_{i = 1}^{n} {( {d ( i )}_{pos / neg} - {μ}_{{d}_{pos / neg }} )}^{2}} & ( Formula 20 )\end{matrix}$$

Here, d represents the distance from the measured value (modulation value) of each patient to the center 1402 or 1403 in the positive modulation region or the negative modulation region. The variable μ_{dpos/neg} represents the mean value of the distance from the measured value of each patient to the center in each modulated region. The variable σ_{dpos/neg} represents the standard deviation of the distance from the measured value of each patient to the center in the modulated region.

The regions 1405 and 1407 are defined by μ_{dpos}+/−σ_{dpos} and μ_{dneg}+/−σ_{dneg}, respectively. The region 1406 is defined by the region between μ_{dneg}+σ_{dneg} and μ_{dpos}−σ_{dpos}. The region 1406 is a region where efficacy cannot be definitively determined (low efficacy, or efficacy is small, if any).

FIG. 14 represents an example in which the current measurement result 1404 falls in the effective region 1405.

As can be understood from the foregoing descriptions, the region 1405 is a region where the presence of efficacy can be definitively determined, whereas the region 1407 is a region where efficacy can be determined as being absent.

### Scatter Chart Sorting Efficacy by Activity-Variability Analysis (Clustering) (Example)

FIG. 15 shows a scatter chart 1500 sorting efficacy by activity-variability analysis (clustering).

The clustering described in FIG. 11 sorts data into a non-modulated region and a modulated region. The non-modulated region includes pre-administration data, and post-administration data determined as having no efficacy. The modulated region is a region that possibly includes healthy individual data, and post-administration data determined as having efficacy. The two regions are separated from each other by a dividing line 1501, and the centers 1502 and 1503 of the distribution data are placed in these regions.

Whether the data falls in the modulated region is determined by confirming sensitivity and specificity, as described in FIG. 11.

The distance 1504 from the pre-administration measurement data (currently measured data) to the dividing line 1501 is calculated. The distance 1505 from the post-administration measurement data (currently measured data) to the dividing line 1501 is also calculated. These distance values become comparison indices between the pre-administration state and the post-administration state. The graph suggests that efficacy is absent when the post-administration measurement data falls in the non-modulated region.

In sorting efficacy by the threshold as above, the standard deviation of amplitudes, and the amplitude difference between task trials are used as feature amounts, as shown in FIG. 11. The threshold also may be determined by other methods such as by using a support vector machine, or by discriminatory analysis or clustering.

In the present embodiment, the position of the patient (subject) of interest in the measurement database 109b is grasped. This enables accurate calculations of efficacy index.

### Relation Between Task Correctness Change and Blood Volume

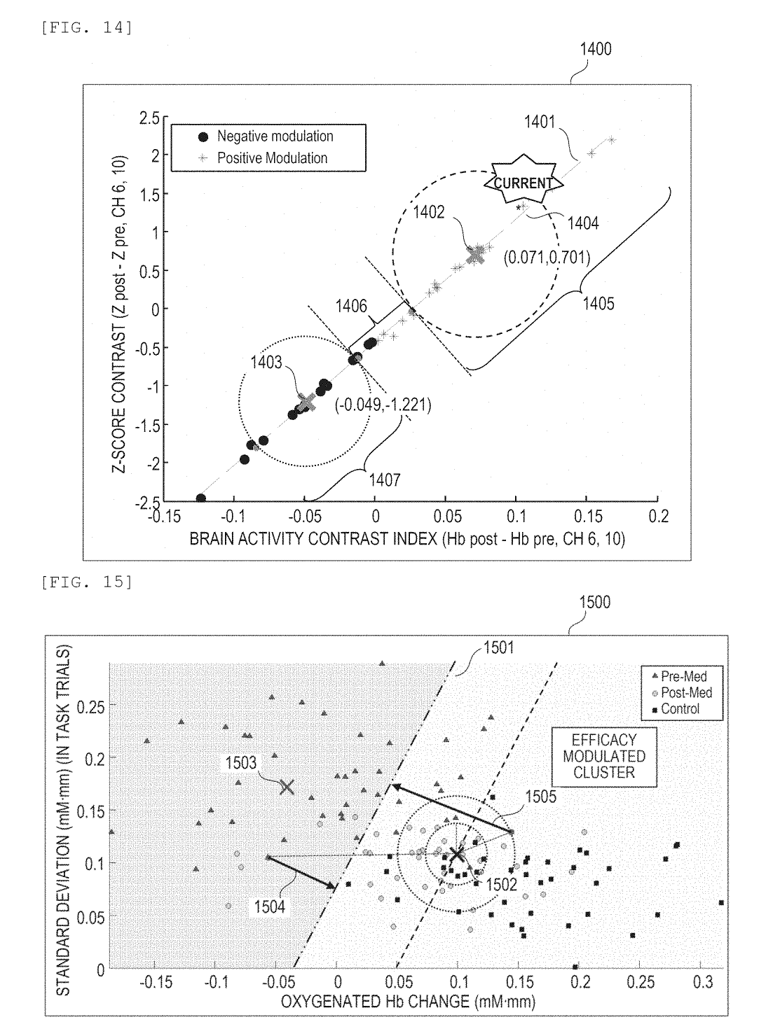

FIG. 16 shows a diagram 1600 representing the relationship between task correctness change and blood volume change. The diagram integrates the biological measurement result with the result of a performed task, and represents another method of determining the presence or absence of efficacy using a technique different from the efficacy index computation described in FIG. 10.

First, the neuromodulation index is calculated for all patients according to formula 1. Changes in the correctness of the task performed by all patients (correct rate after administration−correct rate before administration) are also calculated, according to formula 21.

[Math. 13]

Contrast(i)=Correct rate(i)ₚₒₛₜ−Correct rate(i)ₚᵣₑ (Formula 21)

By setting the correct rate change and the neuromodulation index on X axis and Y axis, respectively, a straight line 1601 can be plotted upon finding the relationship between these. There is no improvement or decline in task result when the correct rate change is 0. There is no improvement or decline in brain activity (average) when the neuromodulation index is 0. In this case, the threshold of the presence or absence of efficacy is 0. Efficacy can be determined as being present when the correct rate change and the neuromodulation index are both larger than 0 (region 1603), and being absent when either of these is smaller than 0 (regions 1604, 1605, and 1606).

In the example shown in FIG. 16, the current measurement result 1602 falls in the first quadrant of the graph 1600, and the current measurement result 1602 can be determined as having efficacy.

### Channel Connectivity

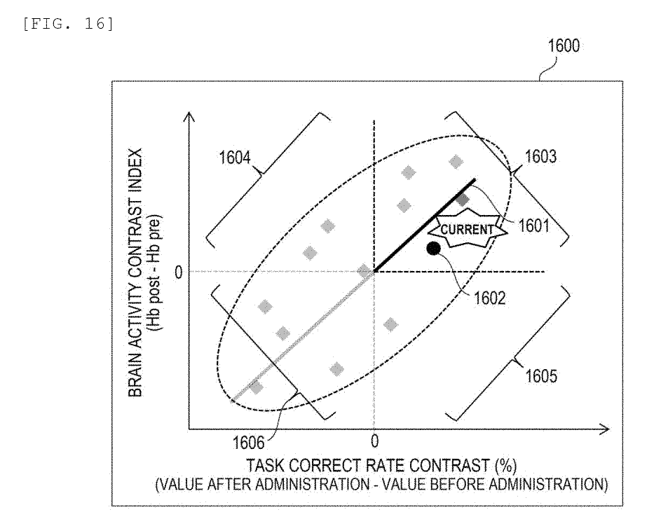

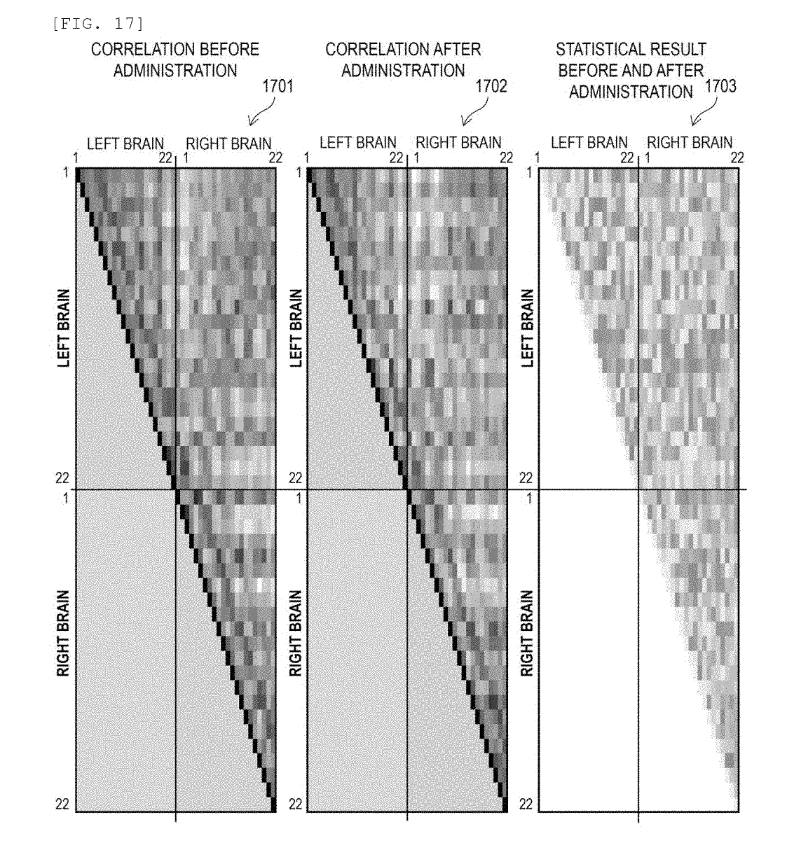

FIG. 17 shows a correlation diagram used to grasp the intensity of the functional connectivity between channels. When using this correlation diagram, a correlation diagram of the functional connectivity of a healthy individual is used as a template.

A correlation 1701 represents the correlation between the probe channels before administration. In the case of the probe placement shown in FIG. 3, the correlation is between channels 1 to 22 attached to each hemisphere (left and right). Darker areas indicate stronger correlations, and can be interpreted as having strong connectivity.

A correlation 1702 represents the correlation between probe channels after administration. A correlation 1703 represents the statistical result before and after administration, and is obtained as the difference of the correlation 1702 and the correlation 1701.

The areas of strong connectivity and strong connection differ for different mental disorders, and the subject can be determined as being normal or potentially having some kind of mental disorder by comparing the connectivity with the template. Specifically, stronger efficacy can be determined as the connectivity after administration (correlation 1702) shares more similarity to the template of a healthy individual, whereas efficacy can be determined as being weak as the similarity decreases. The correlation also varies with the type of administered drug.

The correlation between probe channels can be calculated according to, for example, formulae 22 and 23.

$$\begin{matrix} [ Math . 14 ] & \\ {signal ( i )}_{pre / post / control} = {[ \begin{matrix}{x}_{1 , 1} & … & {x}_{1 , t} \\ ⋮ & ⋱ & ⋮ \\ {x}_{ch , 1} & … & {x}_{ch , t}\end{matrix} ]}_{ch \times {t}_{{( i )}_{pre / post / control}}} & ( Formula 22 ) \\ {ρ ( {x}_{( ch ( 1 ) , t )} , {x}_{( ch ( 2 ) , t )} )}_{pre / post / control} = \frac{1}{t 2 - t 1} {\sum }_{t = t 1}^{t 2} ( \frac{{x}_{( ch ( 1 ) , t ) - {μ}_{{x}_{ch ( 1 )}}}}{{σ}_{{x}_{ch ( 1 )}}} ) ( \frac{{x}_{( ch ( 2 ) , t ) - {μ}_{{x}_{ch ( 2 )}}}}{{σ}_{{x}_{ch ( 2 )}}} ) & ( Formula 23 )\end{matrix}$$

Here, x represents the amplitude of hemoglobin change at each sampling point (t) and measured channel (ch), μ represents the amplitude signal of a single channel, σ represents the standard deviation of the amplitude signal of a single channel, and ρ represents the coefficient of correlation of a signal between two channels in each state (before administration, after administration, and healthy condition).

### Details of Response Prediction Process (Step 811)

FIGS. 18A and 18B are flowcharts explaining details of the response prediction process (step 811) for predicting a response to a future treatment.

### (i) Step 1801

The response prediction program 105b reads a prediction command entered by a doctor or other user (see FIG. 7). The prediction command includes, for example, dose-index relation, multiple linear regression, sigmoid, and ANOVA (Analysis of variance).

### (ii) Step 1802

The response prediction program 105b determines whether the prediction command it has read is dose-index relation. The sequence goes to step 1803 when the command is dose-index relation (Yes in step 1802). When the command is not dose-index relation (No in step 1802), the sequence goes to step 1813.

(iii) Step 1803

The response prediction program 105b recognizes (specifies) the type of performed task, and the type of administered drug in the current measurement.

### (iv) Step 1804

By using the efficacy index command (701 in FIG. 7), the response prediction program 105b reads the data of all patients who have undertaken the same task and had the same drug as those performed or used in the current measurement. The data are read from the measurement database 109b, and an index is calculated for the all patients.

### (v) Step 1805

The response prediction program 105b pairs the calculated index with a dose for each type of administered drug (for example, MPH, and ATX).

### (vi) Step 1806

The response prediction program 105b plots an index versus dose relation (box-plot) for each type of administered drug by setting the index and the dose on Y axis and X axis, respectively.

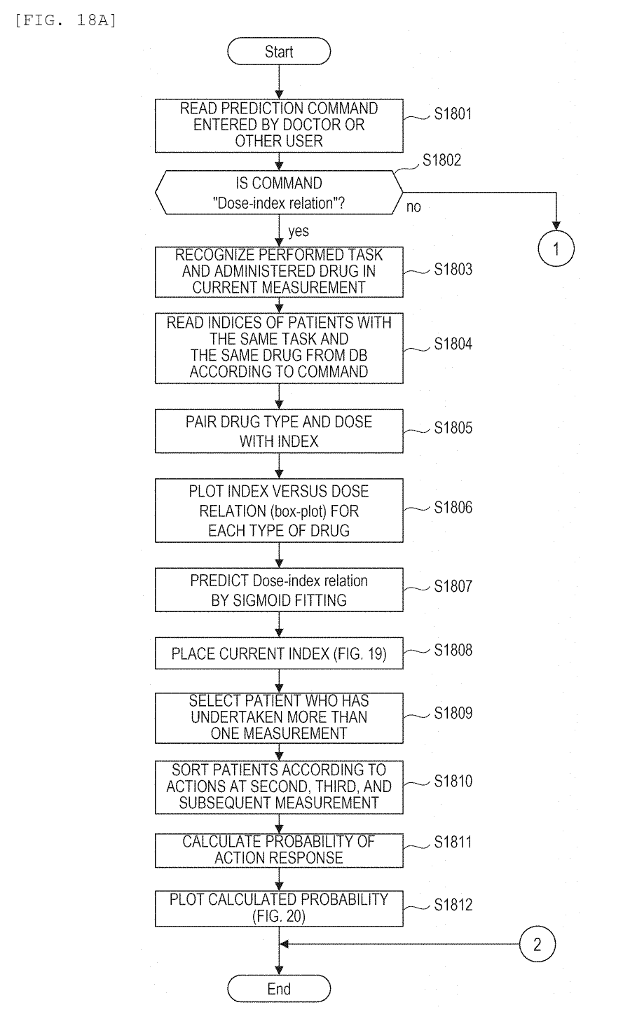

Pre-administration data may be contained as a baseline index, depending on the selected efficacy index analysis. Because the efficacy threshold (the threshold for determining the presence or absence of efficacy) is determined in each index generating method, the threshold also may be plotted (y=threshold (for example, 1904 in FIG. 19)).

(vii) Step 1807

The response prediction program 105b predicts the relation between index and dose (dose-index relation) by sigmoid fitting.

(viii) Step 1808

The response prediction program 105b places the index based on the current measurement, as shown in FIG. 19.

### (ix) Step 1809

In order to predict a future response of a patient by probability analysis concerning various drug treatments, the response prediction program 105b selects from the measurement database 109b the conditions of a similar patient having a measurement count of more than one (data of a patient who has responded to a specific drug treatment).

### (x) Step 1810

The response prediction program 105b sorts the indices of other patients according to the information of the further actions recorded in the measurement database 109b (further actions 215 at the second, third, and any subsequent measurements).

### (xi) Step 1811

The response prediction program 105b calculates the probability of action response using a technique such as Bayesian inference (Bayes' theorem).

(xii) Step 1812

The response prediction program 105b plots the calculated probability of action response (step 1811) in the dose-index relation generated in step 1807 (see FIG. 20).

(xiii) Step 1813

The response prediction program 105b determines whether the prediction command it has read is “sigmoid” or “multiple linear regression”. The sequence goes to step 1814 when the read prediction command is “sigmoid” or “multiple linear regression” (Yes in step 1813). When the read prediction command is neither of “sigmoid” and “multiple linear regression” (No in step 1813), the sequence goes to step 1826.

(xiv) Step 1814

The response prediction program 105b determines whether the measurement count is more than 3. The sequence goes to step 1817 when the measurement count is more than 3 (Yes in step 1814). When the measurement count is 3 or less (No in step 1814), the sequence goes to step 1815. More than three measurement counts are required to ensure a certain level or more of prediction accuracy.

### (xv) Step 1815

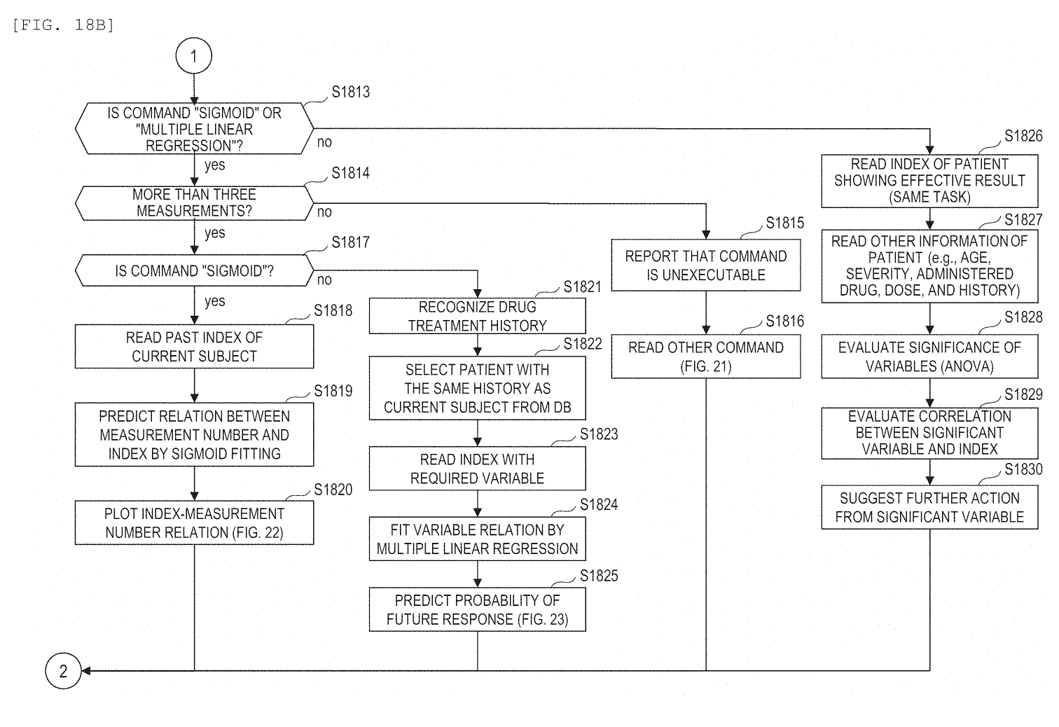

The response prediction program 105b outputs an error report reporting that the read prediction command is not executable (see FIG. 21).

(xvi) Step 1816

The response prediction program 105b waits for input of an instruction from a doctor or other user (pressing of the OK button or reset button) before reading other prediction command (see FIG. 21).

(xvii) Step 1817

The response prediction program 105b determines whether the prediction command it has read is “sigmoid”. The sequence goes to step 1818 when the read prediction command is “sigmoid” (Yes in step 1817). When the prediction command is not “sigmoid” (No in step 1817), the sequence goes to step 1821.

(xviii) Step 1818

The response prediction program 105b reads the past index of the current subject (patient) of interest from the measurement database 109b.

(xix) Step 1819

The response prediction program 105b predicts the relationship between the index read in step 1818, and the measurement number, using sigmoid fitting.

### (xx) Step 1820

The response prediction program 105b plots the relationship between measurement number and index by setting the measurement number and the index on X axis and Y axis, respectively (see FIG. 22).

(xxi) Step 1821

The response prediction program 105b refers to the private database 109a or the measurement database 109b, and recognizes the medication history of the current subject (patient) of interest.

(xxii) Step 1822

The response prediction program 105b selects all patients having the same history as the current subject (patient) of interest.

(xxiii) Step 1823

The response prediction program 105b reads the indices of these patients from the measurement database 109b, together with requested variables such as the type and dose of administered drug (704 in FIG. 7).

(xxiv) Step 1824

The response prediction program 105b fits the relationship between the read variables, using multiple linear regression.

(xxv) Step 1825

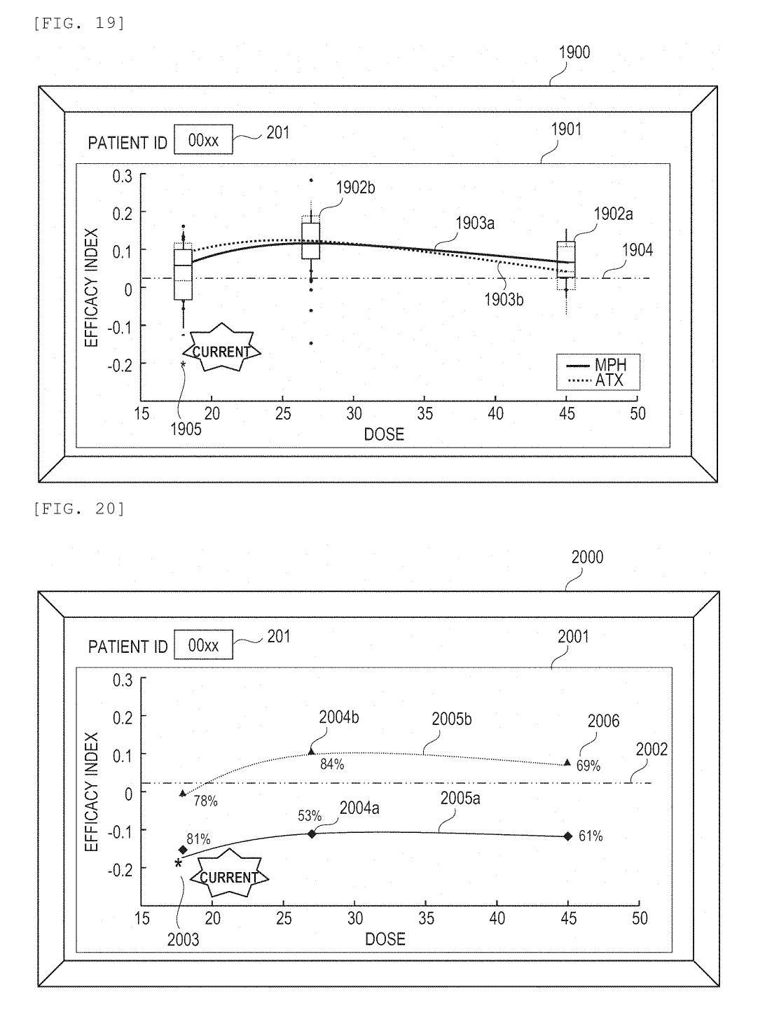

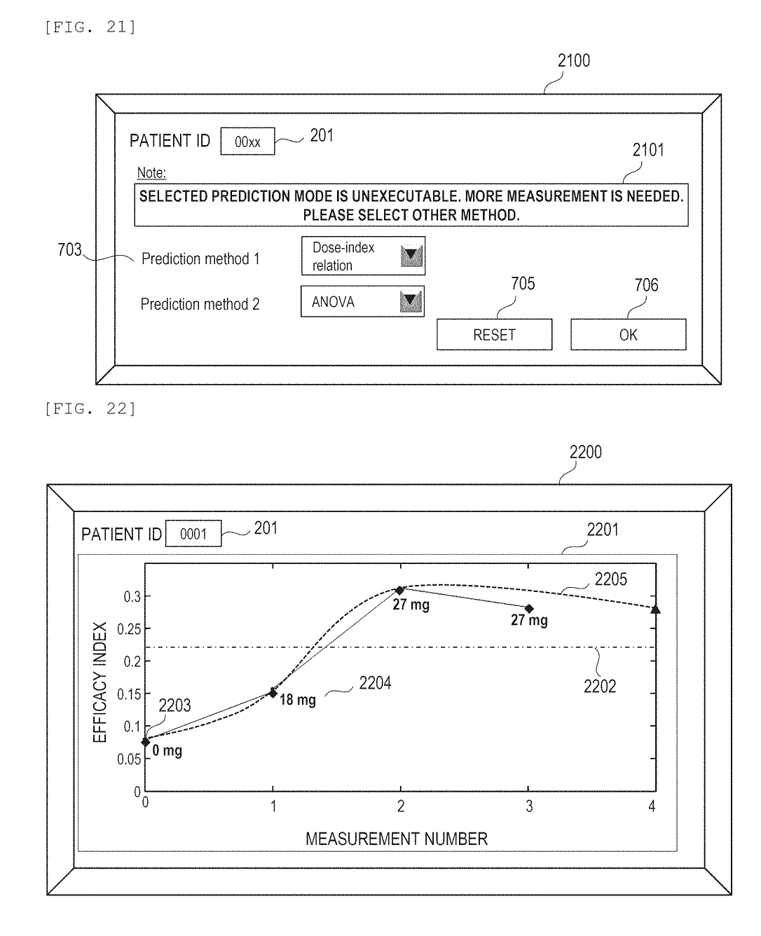

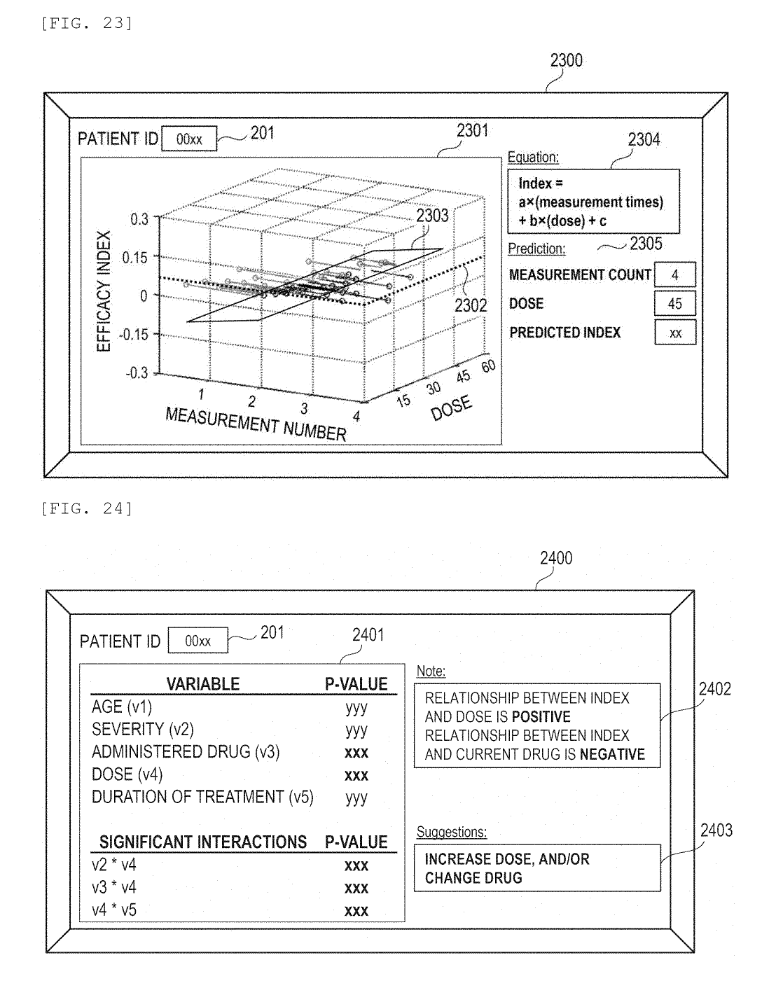

The response prediction program 105b predicts the probability of further response, using the input variables (see FIG. 23).

(xxvi) Step 1826

When the read prediction command is ANOVA (analysis of variance), the response prediction program 105b reads from the measurement database 109b the indices of all patients for which efficacy has been determined in the same task being currently measured for the subject (patient) of interest.

(xxvii) Step 1827

From the private database 109a, the response prediction program 105b reads other information of the patients specified in step 1826 (for example, age, severity, administered drug, dose, and history).

(xxviii) Step 1828

The response prediction program 105b evaluates the significance of the variables (ANOVA).

(xxix) Step 1829

The response prediction program 105b evaluates the correlation between index and significant variable.

(xxx) Step 1830

The response prediction program 105b suggests (presents) a further action from the correlation between index and significant variable obtained in step 1829.

### Relationship Between Dose and Drug-Induced Signal Change

FIG. 19 is a diagram representing a dose-index relation 1900 generated in the response prediction process (S1802 to S1808 in FIG. 18).

The dose-index relation 1900 displays, for example, a patient ID 301 for specifying the patient of interest for response prediction, and a relation 1901 between dose (applied dose) and efficacy index.

The relation 1901 between dose (applied dose) and efficacy index is configured from an index distribution of specific prescriptions (administered drug: for example, MPH, and ATX) expressed as boxplots 1902a and 1902b, predicted sigmoid fitting curves 1903a and 1903b indicating how the efficacy index varies with increasing doses of each administered drug, an efficacy threshold index (relation formula of a straight line with y=threshold) 1904, and an index and dose 1905 of the current measurement.

When the current result falls outside of the upper and lower whiskers of the boxplot of the corresponding administered drug, the result can be interpreted as being different from the patient data measured in the system (FIG. 1), and having a (high) possibility of no efficacy.

The dose-index relation 1900 can assist a doctor or other user to decide a further treatment regimen for the patient of interest.

The sigmoid fitting in FIG. 19 may be determined by using, for example, formula 24.

$$\begin{matrix}[ Math . 15 ] & \\ {S ( dose )}_{MPH / ATX} = \frac{1}{1 + {e}^{- dose}} & ( Formula 24 )\end{matrix}$$

Here, S represents sigmoid fitting for an efficacy index (for example, a modulation index, and a distance index) for different administered drugs (for example, MPH, and ATX) using a dose-dependent variable.

### Display of Probability Analysis Result

FIG. 20 shows the probability generated in the response prediction process (steps 1811 and 1812) plotted in the dose-index relation 2000 (probability analysis result).

As with the case of the dose-index relation 1900, the dose-index relation 2000 is configured from a patient ID 301 for specifying the patient of interest for response prediction, and the relation 2001 between dose (applied dose) and efficacy index.

In FIG. 20, the relation 2001 between dose (applied dose) and efficacy index displays a threshold 2002 for the selected efficacy analysis, a current measurement result 2003, a future response prediction 2004a with a similar prescription, a future response prediction 2004b with a dissimilar prescription, a fitting result 2005a for prediction of an index transition with an increased dose of a similar prescription, a fitting result 2005b for prediction of an index transition with an increased dose of a dissimilar prescription, and an efficacy probability 2006.

The prediction of each index transition for a specific administered drug and dose is shown with the result of a probability analysis for the prediction. This makes it possible to assist a doctor or other user to choose an appropriate further treatment that provides efficacy at higher probability.

The probability of efficacy prediction is calculated according to formula 25 (Bayes' rule).

$$\begin{matrix}[ Math . 16 ] & \\ P ( H \vert E ) = \frac{P ( E \vert H ) \cdot P ( H )}{P ( E )} & ( Formula 25 )\end{matrix}$$

Here, P(H|E) represents the posterior probability of a specific prescription that provides an effective response, P(E|H) represents the probability of an effective response with a specific prescription, P(H) presents the prior probability of an effective response with a specific prescription, and P(E) represents the probability of an effective response unrelated to the prescription. A future response can be predicted by carrying out the probability analysis (Bayesian inference) for each prescription and dose in a selected group (patients having the same conditions as the current patient of interest).

### Exemplary Structure of GUI Reporting Prediction Command Execution Error

FIG. 21 is a diagram representing an exemplary structure of a GUI showing a report output when the response prediction process cannot be executed as instructed by the prediction command (S1815 in FIG. 18). The report in this example is displayed when the measurement count has not reached the specified number.

For example, an error is reported when the measurement count is not high enough to execute the prediction method instructed by the read prediction command, as shown in the comment box 2101 of a report screen 2100.

When presented with such an error report, a doctor or other user can execute other prediction method by choosing a different prediction method (selection of prediction method 703), and pressing the OK button 706. The GUI may be adapted so that the selected prediction method is cancelled by pressing the reset button 705, or that the response prediction process is ended by pressing the reset button 705 while the selection of prediction method 703 is blank.

Relationship between Measurement Number and Efficacy Index FIG. 22 is a diagram representing an example of the relationship 2200 between efficacy index and measurement number generated in the response prediction process (S1818 to S1820 in FIG. 18: sigmoid prediction). Specifically, FIG. 22 shows a transition of drug-induced signal changes according to measurement number and dose, and a regression curve of drug-induced signal changes against measurement number. The sigmoid prediction is executable when the measurement count is higher than 3.

The relationship 2200 between measurement number and efficacy index is configured from, for example, a patient ID 301 for specifying the patient of interest of response prediction, and efficacy index changes 2201 against measurement number.

The efficacy index changes 2201 against measurement number take into consideration pre-administration as a reference (baseline), and display an efficacy threshold 2202 for evaluating the presence or absence of efficacy at each measurement number, a transition of patient's personal measurement index (the first, second, third, and any subsequent measurement) 2203 as a measurement history, a dose 2204, and sigmoid fitting and modeling 2205 for predicting a future response with an quantitative index.

The presence or absence of efficacy can be determined from the parameter of the regression curve. Specifically, efficacy can be determined when the absolute value of the efficacy index is greater than the predetermined value, and when the slope of the regression curve approaches zero. A future response can be predicted with the history of the patient's personal measurement results.

The sigmoid fitting in FIG. 22 may be determined using, for example, formula 26.

$$\begin{matrix}[ Math . 17 ] & \\ {S ( times )}_{MPH / ATX} = \frac{1}{1 + {e}^{- times}} & ( Formula 26 )\end{matrix}$$

Here, S represents sigmoid fitting for an efficacy index (for example, a modulation index, and a distance index) for different administered drugs (for example, MPH, and ATX) using a measurement count-dependent variable.

### Prediction Result by Multiple Linear Regression (Example)

FIG. 23 is a diagram representing an example of the relationship 2300 between the variables generated in the response prediction process (S1821 to S1825 in FIG. 18: prediction by multiple linear regression).

The relationship 2300 between variables is configured from, for example, a patient ID 301 for specifying the patient of interest for response prediction, a relation 2301 between efficacy index and variables, an index formula (linear formula) 2304 obtained by multiple linear regression, and a prediction result 2305. Here, the relation 2301 between efficacy index and variables is represented by the three-dimensional graph. However, the dimensions are determined by the number of variables.

The relation 2301 between efficacy index and variables displays a threshold 2302 for identifying the presence or absence of efficacy in each data, and a correlation 2303 between variables calculated by multiple linear regression.

In the prediction result 2305, a doctor or other user enters a variable (for example, measurement count, or dose) in the variable field, and the variable is applied to the index formula 2304. The system then outputs the calculated index value.

The index formula 2304 may be represented by formula 27.

$$\begin{matrix} [ Math . 18 ] & \\ [ \begin{matrix}{Index}_{( 1 )} \\ ⋮ \\ {Index}_{( n )}\end{matrix} ] = [ \begin{matrix}{Times}_{( 1 )} \\ ⋮ \\ {Times}_{( n )}\end{matrix} ] [ \begin{matrix}{α}_{( 1 )} \\ ⋮ \\ {α}_{( n )}\end{matrix} ] + [ \begin{matrix}{Dose}_{( 1 )} \\ ⋮ \\ {Dose}_{( n )}\end{matrix} ] [ \begin{matrix}{β}_{( 1 )} \\ ⋮ \\ {β}_{( n )}\end{matrix} ] + [ \begin{matrix}{c}_{( 1 )} \\ ⋮ \\ {c}_{( n )}\end{matrix} ] & ( Formula 27 )\end{matrix}$$

Here, Index represents the past efficacy index, Times and Dose represent dependent variables (measurement count and dose, respectively), c is a constant representing the characteristics of changes between administered drugs and between patients, and n represents the number of group data.

### ANOVA Result

FIG. 24 is a diagram representing an example of a displayed ANOVA result. The results for which efficacy was determined for the same task are collected, and subjected to ANOVA (analysis of variance). The ANOVA result is then displayed along with information of the significant interactions between variables (for example, age, severity, administered drug, dose, and duration of treatment).

The ANOVA result display 2400 displays, for example, a patient ID 301 for specifying the patient of interest for response prediction, a P-value (results with smaller P-values are more significant) 2401 representing the significance between variables, a note section 2402 showing the relationship (correlation) between index and each variable, and a suggestion 2403 concerning further actions based on the correlation of variables.

Here, a variable being significant means that varying the variable (for example, varying the dose) has significant impact on brain activity change (index). For example, a P-value of v3× v4 becomes smaller when the drug and dose (v3×v4) produces a large change in brain activity. As an example, efficacy can be determined as being present when the P-value is less than a predetermined significance level (for example, 0.05). Efficacy is absent when the P-value is more than 0.05, and a further action can be presented by suggesting increasing dose.

### Review

(1) In the present embodiment, the drug efficacy evaluation assisting system reads from the memory the measurement information (Hb concentration) of the brain activity of a subject (patient) of interest measured before and after drug administration, calculates brain activity modulation (Hb concentration changes) before and after drug administration in a plurality of measurements, and displays the relationship between measurement number and the brain activity modulation of the subject of interest on a screen of a display device (see FIG. 22). The system reads from the memory the measurement information of the brain activity of a plurality of subjects before and after drug administration, calculates a statistical value of the brain activity modulation of the subjects as a threshold, and also displays the threshold on the display screen. By presenting such information, an objective index about efficacy is presented to a doctor or other user. This enables a doctor or other user to determine the presence or absence of the effect (efficacy) of a drug treatment, and determine whether to continue the similar treatment.

The system may be adapted to also display the dose (applied dose) in each measurement on the screen. This helps more easily understand the relationship between dose and brain activity modulation, and enables more quantitative efficacy evaluations. The system also helps understand the effectiveness of a drug, and enables assisting patients and patient's families in deciding to choose a drug.

The relationship between measurement number and the brain activity modulation of the subject of interest may be expressed and displayed in the form of a regression curve.

(2) In the present embodiment, the drug efficacy evaluation assisting system performs the following processes in response to an input analysis command (see FIG. 7).

(i) A first analysis process that generates drug efficacy evaluation assist information, and presents the information on a screen of a display device according to the relationship between brain activity simple modulation before and after drug administration, and the number of subjects (see FIG. 12).

(ii) A second analysis process that generates drug efficacy evaluation assist information, and presents the information on the screen according to the relationship between the z-score of brain activity before drug administration, and the z-score of brain activity after drug administration (see FIG. 13).

(iii) A third analysis process that generates drug efficacy evaluation assist information, and presents the information on the screen according to the relationship between brain activity simple modulation before and after drug administration, and changes in the z-score of brain activity before and after drug administration (see FIG. 14).

(iv) A fourth analysis process that generates drug efficacy evaluation assist information, and presents the information on the screen by using a predetermined clustering process (see FIG. 15).

Any one of the first to fourth analysis processes may be performed, or more than one of these processes may be performed. The system is not necessarily required to be configured to perform the first to fourth analysis processes, provided that any one of these processes can be performed. This is because objective and quantitative information for determining efficacy can be presented by any of these processes. The system may be adapted to place the data of the patient of interest for evaluation (measurement result) on the drug efficacy evaluation assist information. In this way, a doctor or other user is able to objectively and more accurately determine the presence or absence of the efficacy of the drug treatment given to a patient. The system also helps understand the effectiveness of a drug, and enables assisting patients and patient's families in deciding to choose a drug.

The system determines the presence or absence of efficacy according to the relationship between the placed data of the patient of interest, and the drug efficacy evaluation information, and presents the result of determination on the screen (see FIG. 9). In this way, a doctor or other user is able to grasp the presence or absence of efficacy without interpreting a graph or the like to find whether there is efficacy.

Specifically, the first analysis process calculates at least brain activity modulation values for all patients before and after administration, and a distribution of the brain activity modulation values before and after administration, and uses the statistical value (mean value of the modulation values) from the distribution calculation as a threshold. The distribution and the threshold are then displayed on a screen of a display device (see FIG. 12). Efficacy can be determined to be present when the current measurement result of the patient of interest lies on the right of the threshold (mean value). The first analysis process enables grasping the relative position of a specific patient in all patients, and objectively determining the presence or absence of efficacy.

In the second analysis process, the relationship in the z-scores of brain activity before and after administration is calculated as a threshold line by linear regression computation in the absence of brain activity modulation before and after administration, and the threshold line is displayed (see FIG. 13). Basically, efficacy is determined to be present when the data lies in a region above the threshold line, and absent when the data lies in a region below the threshold line. A region in the vicinity of the threshold line may be defined as a region where efficacy determination is not possible. In this way, the presence or absence of efficacy can be more accurately and definitively determined.

The third analysis process calculates a modulation value of brain activity for all subjects before and after administration, and a z-score contrast representing a change in the z-score of brain activity of all subjects before and after drug administration, and displays the relationship between the brain activity modulation value before and after administration and the z-score contrast on the screen (FIG. 14). In this way, the presence or absence of efficacy can be more accurately and definitively determined, as in the second analysis process.

The fourth analysis process calculates the mean value of brain activity modulation of healthy individuals who have undertaken the predetermined task multiple times (here and below, modulation in an activity interval), the mean value of brain activity modulation of each subject patient who has undertaken the predetermined task multiple times before drug administration, and the mean value of brain activity modulation of each subject patient who has undertaken the predetermined task multiple times after drug administration. The process also calculates a dispersion variable of brain activity modulation for each healthy individual who has undertaken the predetermined task multiple times, a dispersion variable of brain activity modulation for each patient who has undertaken the predetermined task multiple times (before administration), and a dispersion variable of brain activity modulation for each patient who has undertaken the predetermined task multiple times (before administration). The mean value of brain activity modulation, and the dispersion variable of brain activity modulation are set on X and Y axes. A combination of the mean value and the dispersion value of each healthy individual, and a combination of the mean value and the dispersion value of each patient before and after administration are placed on the X-Y plane. The placed data are then subjected to a predetermined clustering process (for example, k-means clustering) to separate the placed data into two regions at a threshold line (see FIG. 15). By indicating the positions of each healthy individual and each patient (subject) in all data, the presence or absence of efficacy can be accurately evaluated.

(3) In the present embodiment, the drug efficacy evaluation assisting system performs the following processes in response to an input prediction command (see FIG. 7).

(i) A first prediction process that generates efficacy predicting information for a plurality of subjects (patients) according to the relationship between dose and the value of brain activity modulation before and after drug administration, and presents the information on a screen of a display device.

(ii) A second prediction process that generates efficacy predicting information for a patient of interest according to the relationship between the measurement number of brain activity, and the value of brain activity modulation before and after administration, and presents the information on the screen.

(iii) A third prediction process that generates efficacy predicting information for a plurality of patients according to the relationship between a variable contained in the prediction command, and the value of brain activity modulation before and after administration, and presents the information on the screen.

(iv) A fourth prediction process that evaluates the significance between variables using ANOVA (analysis of variance), generates efficacy predicting information according to the evaluation result, and presents the result on the screen.

This enables a doctor or other user to readily decide a further action (for example, end treatment, continue treatment, change prescription, or change dose) for a specific patient.

In the first and second prediction processes, the statistical value (for example, the mean value) of the brain activity modulation of a plurality of patients is used as a threshold, and the threshold is presented together with the efficacy predicting information. This makes it possible to find the dose or the frequency of administration of time when the data is above the threshold.

In the first and second prediction processes, the data (measurement result) of a patient of interest for evaluation is placed on the drug efficacy evaluation assist information. In this way, a doctor or other user is able to make an overall determination as to the future outcome of a drug treatment corresponding to the current measurement (how much dose is needed to provide efficacy), or whether the current drug should be continuously used.

(4) The present invention also can be achieved by software program codes intended to provide the functions of the embodiment. In this case, a storage medium storing such program codes is provided for a system or a device, and a computer (or a CPU or MPU) of the system or the device reads the program codes stored in the storage medium. In this case, the functions of the embodiment are provided by the program codes themselves read from the storage medium, and the program codes and the storage medium storing the program codes constitute the present invention. The storage medium supplying the program codes may be, for example, a flexible disc, a CD-ROM, a DVD-ROM, a hard disc, an optical disc, a magneto-optical disc, a CD-R, a magnetic tape, a nonvolatile memory card, or a ROM.

The functions of the embodiment also may be provided by all or part of a process actually performed by, for example, the operating system (OS) running on a computer under the commands of the program codes. The functions of the embodiment also may be provided by all or part of a process actually performed by, for example, a CPU of a computer according to the commands of the program codes written into memory of the computer from a storage medium.

Software program codes intended to provide the functions of the embodiment may be delivered via a network, and stored in a hard disc, memory, or some other storage mean of a system or a device, or in a storage medium such as CD-RW, and CD-R. For use, a computer (or a CPU or MPU) of the system or the device may execute the program codes after reading it from the storage means or storage medium.

Finally, it is to be understood that the processes and the techniques described herein, in essence, have no association with a particular device, and can be implemented by any suitable combination of components. A wide range of all-purpose devices can be used according to the teaching of the invention described herein. It may be beneficial to construct a device designed specifically for the execution of the steps of the methods described herein. Various forms of invention are possible by appropriately combining the different constituting elements disclosed in the embodiment. For example, some of the constituting elements described in the embodiment may be omitted. It is also possible to appropriately combine the constituting elements of different embodiments. While the present invention has been described in relation to specific examples, the descriptions above serve solely to illustrate the embodiment of the invention, and do not limit the invention in any respect. A person ordinary skilled in the art will understand that there are many combinations of hardware, software, and firmware that are suitable for implementing the invention. For example, the software may be implemented with a wide range of programs or script languages, including an assembler language, C/C++, perl, Shell, PHP, and Java®.

The control lines and information lines described in the foregoing embodiment are what are considered to be necessary for the purpose of explanation, and do not necessarily represent the all control lines and information lines of a product. All configurations may be interconnected to one another.

As is evident to a person having common knowledge in the art, other implementations of the invention will be apparent from the specification of the invention and the discussions of the embodiment disclosed herein. The specification and the specific examples merely represent typical examples, and the scope and the spirit of the invention lie in the appended claims below.

### REFERENCE SIGNS LIST

1 Drug efficacy evaluation assisting system
100 Evaluation device
101 Input device
102 Processing unit
103 Information processing program
104 Data preprocessing program
105 Drug efficacy program
105a Drug efficacy index/coefficient program
105b Response prediction program
106 Biological measurement unit
107 Task management unit
107a Task output unit
107b Recorder
108 Display device
109 Memory
109a Private database
109b Measurement database
109c Analysis parameter database
110 Output device
111 Input device
201 Patient ID
202 Patient name
203 Patient birthday
204 Patient gender
205 Medication history
206 Measurement date
207 Task
208 Prescription
209 Applied dose
210 Extracted signal
211 Response time
212 Task correct rate
213 Rating scale
214 Diagnosis result
215 Further action
216 Motion elimination
217 High-pass filter coefficient
218 Low-pass filter coefficient
219 Smoothing coefficient
220 Subject of noise correction
221 Suggestive region of interest
222 Activity interval
300 Probe
301 Optical sources
302 Detectors
303 Measurement point channel
500 Channel selecting display screen
501 Biometric signal display region
502 Hemoglobin type selecting button
503 Measurement status selecting button
504 Cerebral hemisphere selecting button
601 Selected channel
602 Selected signal display region
603 Indicated activity interval (stimulus period)
700 Analysis-prediction command input GUI
701 Selection of efficacy index (Efficacy index)
702 Selection of activity interval
703 Selection of prediction method
704 Selection of option variable
705 Reset button
706 OK button
900a-c Efficacy analysis result list display
901 Measurement count
902 Efficacy index
903 Efficacy status
904 Medication type
905 Dose
906a Further treatment, “End measurement”
906b Further treatment, “Continue measurement”
906c Further treatment, blank
907 OK button
908 Prediction button
1200 Graph representing efficacy index of Hb change (simple modulation)
1201 Normal distribution curve
1202 Threshold
1203 Index value
1300 Scatter chart sorting efficacy by Hb change (z-score)
1301 Regression line
1302 Mean distance line in positive modulation region
1303 Mean distance line in negative modulation region
1304 Current measurement result
1305 Effective region
1306 Region where definitive efficacy determination is not possible
1307 Ineffective region
1308 Standard deviation
1309 Standard deviation
1400 Scatter chart sorting efficacy by Hb change (z-score contrast)
1401 Line for measurement results
1402 Center of positive modulation region
1403 Center of negative modulation region
1404 Current measurement result
1405 Effective region
1406 Region where definitive efficacy determination is not possible
1407 Ineffective region
1500 Scatter chart sorting efficacy by activity-variability analysis (clustering)
1501 Dividing line
1502 Center of distribution data (after administration/healthy individual)
1503 Center of distribution data (before administration)
1504 Distance (before administration)
1505 Distance (after administration)
1600 Relationship between task correctness change and blood volume change
1601 Straight line
1602 Current measurement result
1603 Effective region
1604 Ineffective region
1605 Ineffective region
1606 Ineffective region
1701 Pre-dosing correlation
1702 Post-dosing correlation
1703 Statistical result before and after administration
1900 Relationship between dose and drug-induced signal change
1901 Relationship between dose (applied dose) and efficacy index
1902a boxplot (drug 1)
1902b boxplot (drug 2)
1903a Predicted sigmoid fitting curve (drug 1)
1903b Predicted sigmoid fitting curve (drug 2)
1904 Efficacy threshold index
1905 Current measurement result
2000 Result of probability analysis
2001 Relationship between dose (applied dose) and efficacy index
2002 Threshold for efficacy analysis
2003 Current measurement result
2004a Future response prediction (drug 1)
2004b Future response prediction (drug 2)
2005a Predicted fitting result (drug 1)
2005b Predicted fitting result (drug 2)
2006 Efficacy probability
2100 Exemplary structure of GUI reporting prediction command execution error
2101 Comment box
2200 Relationship between measurement number and efficacy index
2201 Efficacy index changes against measurement number
2202 Efficacy threshold
2203 Changes of patient's personal measurement index as measurement history
2204 Applied dose
2205 Sigmoid fitting and modeling
2300 Relationship between variables
2301 Relationship between efficacy index and variables
2302 Efficacy threshold
2303 Correlation between variables
2304 Index formula (linear formula)
2305 Prediction result
2400 ANOVA result
2401 P-value representing significance
2402 Note section indicating relationship (correlation) between index and each variable
2403 Suggestions concerning actions

1. A drug efficacy evaluation assisting system for assisting evaluation of the efficacy of a drug treatment on a subject of interest,
the system comprising:
a processor for reading and executing various programs necessary for drug efficacy evaluation; and
a memory for storing a processing result generated by the processor, and various data,
wherein the memory stores brain activity information measured for a plurality of subjects including the subject of interest before and after drug administration, the brain activity information being stored with corresponding measurement numbers, and
wherein the processor executes:
a process of reading from the memory the brain activity information measured for the subject of interest before and after administration, and calculating modulation of the brain activity before and after administration for each of the measurement numbers, and
a process of displaying a relationship between the measurement number and the brain activity modulation of the subject of interest on a screen of a display device.

2. The drug efficacy evaluation assisting system according to claim 1, wherein the processor executes:
a process of reading from the memory the brain activity information measured for the plurality of subjects before and after drug administration, and calculating a statistical value of the brain activity modulation of the plurality of subjects as a threshold, and
a process of displaying the threshold on the screen together with the relationship between the measurement number and the brain activity modulation of the subject of interest.

3. The drug efficacy evaluation assisting system according to claim 1, wherein the processor further executes a process of calculating a regression curve representing changes of the brain activity modulation against the measurement number, and displaying the regression curve on the screen.

4. The drug efficacy evaluation assisting system according to claim 1, wherein the processor further executes a process of also displaying a dose for the measurement number on the screen.

5. A drug efficacy evaluation assisting system for assisting evaluation of the efficacy of a drug treatment on a subject of interest,
the system comprising:
a processor for reading and executing various programs necessary for drug efficacy evaluation; and
a memory for storing a processing result generated by the processor, and various data,
wherein the memory stores brain activity information measured for a plurality of subjects including the subject of interest before and after drug administration, and
wherein the processor reads the measured brain activity information from the memory in response to an input analysis command, and executes:
(i) a first analysis process that generates drug efficacy evaluation assist information, and presents the information on a screen of a display device according to a relationship between brain activity simple modulation and the number of subjects before and after drug administration;
(ii) a second analysis process that generates drug efficacy evaluation assist information, and presents the information on the screen according to a relationship between a z-score of brain activity before drug administration and a z-score of brain activity after drug administration;
(iii) a third analysis process that generates drug efficacy evaluation assist information, and presents the information on the screen according to a relationship between brain activity simple modulation before and after drug administration, and changes in the z-score of brain activity before and after drug administration; or
(iv) a fourth analysis process that generates drug efficacy evaluation assist information, and presents the information on the screen by using a predetermined clustering process.

6. The drug efficacy evaluation assisting system according to claim 5, wherein the processor further executes:
a process of placing data based on the measured information of the subject of interest for evaluation on the drug efficacy evaluation assist information; and
a process of determining the presence or absence of efficacy according to a relationship of the placed data of the subject of interest and the drug efficacy evaluation assist information, and presenting the result of the determination on the screen.

7. The drug efficacy evaluation assisting system according to claim 5, wherein the processor in executing the first analysis process calculates at least brain activity modulation values for all subjects before and after drug administration, and a distribution of the brain activity modulation values before and after drug administration, uses a statistical value from the distribution calculation as a threshold, and displays the distribution and the threshold on the screen of the display device.

8. The drug efficacy evaluation assisting system according to claim 5, wherein the processor in executing the second analysis process calculates a relationship in the z-score of brain activity before and after drug administration as a threshold line by linear regression computation in the absence of brain activity modulation before and after drug administration, and displays the threshold line as the drug efficacy evaluation assist information on the screen.

9. The drug efficacy evaluation assisting system according to claim 5, wherein the processor in executing the third analysis process calculates a modulation value of brain activity for all subjects before and after administration, a z-score of brain activity for all subjects before drug administration, a z-score of brain activity for all subjects after drug administration, and a z-score contrast representing a z-score change before and after drug administration, and displays a relationship between the brain activity modulation value before and after administration and the z-score contrast on the screen of the display device.

10. The drug efficacy evaluation assisting system according to claim 5,
wherein the memory also stores brain activity information measured for a plurality of healthy individuals, and
wherein the processor in executing the fourth analysis process calculates:
the mean value of brain activity modulation of healthy individuals who have undertaken a predetermined task multiple times, using the brain activity information measured for the plurality of healthy individuals, the mean value of brain activity modulation of subjects who have undertaken the predetermined task multiple times before drug administration, using the brain activity information measured for the plurality of subjects before drug administration, and the mean value of brain activity modulation of subjects who have undertaken the predetermined task multiple times after drug administration, using the brain activity information measured for the plurality of subjects after drug administration; and
a dispersion variable of brain activity modulation for each healthy individual who has undertaken the predetermined task multiple times, a dispersion variable of brain activity modulation for each subject who has undertaken the predetermined task multiple times before administration, and a dispersion variable of brain activity modulation for each subject who has undertaken the predetermined task multiple times after administration, and
wherein the processor sets the mean value of brain activity modulation, and the dispersion variable of brain activity modulation on X and Y axes, and places a combination of the mean value and the dispersion variable of each healthy individual, and a combination of the mean value and the dispersion variable of each subject before and after drug administration on the X-Y plane, and
wherein the processor applies a predetermined clustering process to the data placed on the X-Y plane to separate the placed data on the X-Y plane into two regions at a threshold line.

11. The drug efficacy evaluation assisting system according to claim 5, wherein the processor reads the measured brain activity information from the memory in response to an input prediction command, and executes:
(i) a first prediction process that generates efficacy predicting information for the plurality of subjects according to a relationship between drug dose and the value of brain activity modulation before and after drug administration, and presents the information on the screen of the display device;
(ii) a second prediction process that generates efficacy predicting information for the subject of interest according to a relationship between the measurement number of brain activity, and the value of brain activity modulation before and after drug administration, and presents the information on the screen;
(iii) a third prediction process that generates efficacy predicting information for the plurality of subjects according to a relationship between a variable contained in the prediction command, and the value of brain activity modulation before and after drug administration, and presents the information on the screen; or
(iv) a fourth prediction process that evaluates the significance between variables using analysis of variance, generates efficacy predicting information according to the evaluation result, and presents the result on the screen.

12. The drug efficacy evaluation assisting system according to claim 11,
wherein the processor in the first and the second prediction process obtains a statistical value using the brain activity information measured for the plurality of subjects before and after the drug administration, uses the statistical value of the brain activity modulation of the plurality of subjects as a threshold, and presents the threshold on the screen together with the efficacy predicting information.

13. The drug efficacy evaluation assisting system according to claim 11,
wherein the processor in the first and the second prediction process places data based on the measured information of the subject of interest for evaluation on the drug efficacy evaluation assist information.

14. The drug efficacy evaluation assisting system according to claim 11,
wherein the processor further presents a command input display for entry of the analysis command and the prediction command on the screen of the display device, and executes any of the first to fourth analysis processes, and/or any of the first to fourth prediction processes according to the entered content.

15. A drug efficacy evaluation assist information presenting method for presenting information for assisting evaluation of the drug efficacy of a drug treatment on a subject of interest,
the method using a processor that reads and executes various programs necessary for drug efficacy evaluation, and comprising:
the processor reading brain activity information measured for the subject of interest before and after drug administration from a memory storing brain activity information measured for the plurality of subjects including the subject of interest before and after drug administration, the memory storing the brain activity information with corresponding measurement numbers, and the processor calculating modulation of the brain activity before and after drug administration for each of the measurement numbers;
the processor reading from the memory the brain activity information measured for the plurality of subjects before and after drug administration, and calculating a statistical value of modulation of the brain activity of the plurality of subject as a threshold; and
the processor displaying a relationship between the measurement number and the brain activity modulation of the subject of interest and the threshold on a screen of a display device.
